(19) United States
(12) Patent Application Publication
(10) Pub. No.: US 2014/0205582 A1
(43) Pub. Date: Jul. 24, 2014

(54) MEGAKARYOCYTE PROGENITOR CELLS FOR PRODUCTION OF PLATELETS

(71) Applicants: Holger Karsunky, Redwood City, CA (US); Chanawan Chanaukul, San Carlos, CA (US); Swapna Pantuganti, San Carlos, CA (US)

(72) Inventors: Holger Karsunky, Redwood City, CA (US); Chanawan Chanaukul, San Carlos, CA (US); Swapna Pantuganti, San Carlos, CA (US)

(73) Assignee: CELLERANT THERAPEUTICS, INC., San Carlos, CA (US)

(21) Appl. No.: 14/130,894

(22) PCT Filed: Jul. 6, 2012
(86) PCT No.: PCT/US12/45781

§ 371 Date: Mar. 28, 2014

Related U.S. Application Data
(60) Provisional application No. 61/504,972, filed on Jul. 6, 2011.

(51) Int. Cl. C12N 5/078; A61K 35/14

(52) U.S. Cl. CPC C12N 5/0644; A61K 35/19
USPC 424/93.72; 435/372

(57) ABSTRACT

Provided herein are methods of generating megakaryocyte lineage cells from hematopoietic stem cells in the absence of feeder cells and serum. The megakaryocyte progenitor cells (MKPs) generated as described result in rapid production of significant numbers of platelets when administered in vivo.

### STATEMENT AS TO RIGHTS TO INVENTIONS MADE UNDER FEDERALLY SPONSORED RESEARCH AND DEVELOPMENT

This invention was made with Government support under Grant Nos.IRC1 AI080314-01 and -01S1, awarded by the National Institutes of Health and National Institute of Allergy and Infectious Diseases. The Government has certain rights in this invention.

### BACKGROUND OF THE INVENTION

Platelets are essential to the blood clotting process, and are needed to limit leakage of erythrocytes from blood vessels. An abnormally low level of platelets results in an increased risk of bleeding, and can result in spontaneous bleeding when platelet levels drop below a critical level. Thromobocytopenia, a disorder defined by abnormally low platelet levels, can arise from impaired platelet production and/or increased rate of removal.

Platelet transfusion can be used to treat thromobocytopenia, and can be effective in reducing serious bleeding problems associated with low platelet levels. Platelets from MHC-matched donors are typically used to minimize adverse immune response to the donor platelets. The association between infections and thrombocytopenia, however, suggests that neutropenia (reduced white blood cells, particularly neutrophils) complicate thrombocytopenia, requiring more frequent transfusions in patients with both conditions. In addition, use of G-CSF therapy for treating neutropenia is contraindicated for thrombocytopenia because of accelerated platelet destruction correlated with G-CSF administration.

While platelet transfusion for thromobocytopenia is still carried out, there are risks associated with infection and graft rejection. In addition, platelets are not amenable to storage, e.g., cryopreservation, and thus are often not available in sufficient amounts. Thrombocytopenia has also been treated, with modest effect, by administration of thrombopoietin (TPO) or IL-11. TPO does not promote platelet shedding from megakaryocytes, and thus does not immediately increase platelet levels (Ito et al. (1996) Br. J. Haematol. 94:387). IL-11 (Neumega®) affects many different tissue types, and thus can result in side effects (see, e.g., the product insert available at the following website: labeling.pfizer.com/showlabeling.aspx?id=500).

The compositions and methods described herein provide an easily stored population of non-immunogenic megakaryocyte progenitor cells (MKPs) that can be used to generate platelets in vivo.

### BRIEF SUMMARY OF THE INVENTION

Provided herein are compositions and methods for generating a population of megakaryocyte progenitor cells that rapidly produce high numbers of platelets in vivo.

In some embodiments, the invention provides an isolated population of cells comprising CD34+CD41+ megakaryocyte progenitor cells (MKPs) wherein said population of cells has platelet generating activity. In some embodiments, the cells are human cells. In some embodiments, the cells are derived from more than one individual (a plurality of individuals, e.g., at least 2, 3, 4, 5, 6, 7, 8, 9, 10, or more). In some embodiments, the cells are mismatched at one or more MHC (or HLA) loci.

In some embodiments, the population of cells comprise at least 30%, 40, 50, 60, 70, 80, 90 or higher percentage of CD34+CD41+ MKPs. In some embodiments, at least 50% of the CD34+CD41+ MKPs are CD184+ (e.g., 55, 60, 70, 75, 80, 85% or higher percentage CD184+). In some embodiments, less than 75% of the CD34+CD41+ MKPs are CD42a+ (e.g., less than 60, 55, 50, 45, 40, 35, 30, 25, 20, 10, 5% or lower percentage of CD42a+). In some embodiments, the population of cells lacks significant numbers of B and/or T cells (e.g., less than 5, 4, 3, 2, or 1% B and/or T cells, or undetectable levels of B and/or T cells). In some embodiments, the MKP cells are separated from the population of cells, e.g., forming an enriched MKP cell population. In some embodiments, the MKP cells are separated based on cell surface expression of CD34, CD41, and/or CD 184.

In some embodiments, administration of 5×10⁶ cells from the population of cells into an immunocompromised mouse results in at least 10⁶ exogenous (e.g., human) platelets/ml blood, e.g., at 14 days post-administration. In some embodiments, the administration of 5×10⁶ cells results in at least 5×10⁶, 10⁷, 2×10⁷, 5×10⁷, 7.5×10⁷ or 10⁸ exogenous (e.g., human) platelets/ml blood 14 days post-administration into the immunocompromised mouse. In some embodiments, the administration of 5×10⁶ cells results in at least 10⁶, 2×10⁶, 5×10⁶, 7.5×10⁶, or 10⁷ exogenous (e.g., human) platelets/ml blood 6 weeks post-administration into the immunocompromised mouse (e.g., 7, 8, 9, or 10 weeks post-administration).

In some embodiments, the population of cells can generate platelets in vitro, e.g., in media comprising SCF or an SCF analog; TPO or a TPO analog; and/ or heparin (see, e.g.

Baldiuni et al. (2011) PLoSONE 6:e21015; Choi et al. (1995) Blood 85:402). In some embodiments, a population of cells comprising 10⁵-10⁶ MKP cells can generate at least 10⁴-10⁶ platelets in vitro after 4-8 days of culture.

In some embodiments, the population of cells has a megakaryocyte colony formation potential of at least 10%, e.g., at least 12%, 15%, 17%, 20%, 25% or higher. In some embodiments, the population of cells has a megakaryocyte colony formation potential of at least 10% after cryopreservation. In some embodiments, the MKPs further differentiate into CD34^{neg}CD41+CD42a+ megakaryocytes (e.g., as measured by an in vitro Megacult® assay, or detected in vivo after administration into an immunocompromised mouse). In some embodiments, when administered to an immunocompromised mouse, the MKPs engraft in the bone marrow.

In some embodiments, the population of cells is cryopreserved. In some embodiments, the cells retain function post-cryopreservation. That is, the cells can generate platelets or engraft in the bone marrow (as described herein) when administered into an immunocompromised mouse, or can further differentiate into megakaryocytes. In some embodiments, the population of cells retains at least 60, 70, 75, 80, 85, 90, 95, 100% or higher percent function post-cryopreservation compared to the population of cells pre-cryopreservation.

In some embodiments, further provided are pharmaceutical compositions comprising the population of cells described above and a pharmaceutical excipient (e.g., saline solution, buffered saline solution, etc.). In some embodiments, the pharmaceutical composition can be cryopreserved for storage, e.g., prior to administration. In some embodiments, the pharmaceutical composition is administered to an individual, e.g., a thrombopoietic individual, to generate platelets in the individual.

Further provided are methods for preparing the above population of cells that comprises CD34+CD41+ MKPs. In some embodiments, the method comprises culturing CD34+ hematopoietic stem cells (HSCs) in media comprising thrombopoietin (TPO) or a TPO analog and IL-1α under conditions that permit the CD34+ HSCs to generate a population of cells comprising CD34+CD41+ MKPs, wherein the MKPs have platelet generating activity. In some embodiments, the method comprises culturing CD34+ hematopoietic stem cells (HSCs) in media comprising thrombopoietin (TPO) or a TPO analog; IL-1α and/ or IL-1β; and IL-9 under conditions that permit the CD34+ HSCs to generate a population of cells comprising CD34+CD41+ MKPs, wherein the MKPs have platelet generating activity. In some embodiments, the method comprises culturing CD34+ hematopoietic stem cells (HSCs) in media comprising thrombopoietin (TPO) or a TPO analog; IL-1α and/or IL-1β; IL-9; and HSA under conditions that permit the CD34+ HSCs to generate a population of cells comprising CD34+CD41+ MKPs, wherein the MKPs have platelet generating activity. In some embodiments, the method comprises culturing CD34+ hematopoietic stem cells (HSCs) in media comprising thrombopoietin (TPO) or a TPO analog; IL-1α or IL-1β; IL-9; and HSA under conditions that permit the CD34+ HSCs to generate a population of cells comprising CD34+CD41+ MKPs, wherein the MKPs have platelet generating activity.

In some embodiments, the TPO analog is a TPO mimetic. In some embodiments, the media comprises TPO or TPO analog at a concentration of 0.1-100 nM, 1-50 nM, 5-20 nM, 1-25 nM, 5-50 nM, or about 10 nM. In some embodiments, the media comprises IL-1α. In some embodiments, the media comprises IL-1α and/or IL-1β at a concentration of 1-200 ng/ml, 1-100 ng/ml, 1-50 ng/ml, 0.5-20 ng/ml, 5-20 ng/ml, or about 10 ng/ml of IL-1α and/or IL-1β. In some embodiments, where IL-9 is included, the media comprises 1-200 ng/ml, 1-100 ng/ml, 10-100 ng/ml, 10-200 ng/ml, 50-100 ng/ml, or about 60 ng/ml of IL-9. In some embodiments, where HSA is included, the media comprises 0.1-5%, 0.2-4%, 0.5-2%, 0.25-2.5%, 0.5-1.5% or about 1% HSA.

In some embodiments, the culturing is in agitation conditions (e.g., in shaker flasks). In some embodiments, the culturing is started with at least 10⁷ HSCs, e.g., at least any one of 10⁸, 10⁹ or 10¹⁰ HSCs. In some embodiments, the culturing is in static conditions, e.g., in the absence of HSA. In some embodiments, the culture lacks feeder cells. In some embodiments, the media lacks significant or detectable serum (i.e., serum is not added).

In some embodiments, the HSCs are obtained from more than one (a plurality) of individuals. In some embodiments, the HSCs are human. In some embodiments, the HSCs (and MKPs) are mismatched at one or more MHC (or HLA) loci. In some embodiments, the HSCs are obtained from cord blood. In some embodiments, the HSCs are obtained from peripheral blood (e.g., from mobilized peripheral blood). In some embodiments, the HSCs are obtained from induced pluripotent stem cells. In some embodiments, the HSCs are obtained from placenta. In some embodiments, the HSCs are obtained from a combination of sources (e.g., multiple tissues or multiple individuals).

In some embodiments, administration of 5×10⁶ cells from the population of cells into an immunocompromised mouse results in at least 10⁶ exogenous (e.g., human) platelets/ ml blood 14 days post-administration. In some embodiments, the administration of 5×10⁶ cells results in at least 5×10⁶, 10⁷, 2×10⁷, 5×10⁷, 7.5×10⁷ or 10⁸ exogenous (e.g., human) platelets/ml blood 14 days post-administration into the immunocompromised mouse. In some embodiments, the administration of 5×10⁶ cells results in at least 10⁶, 2×10⁶, 5×10⁶, 7.5×10⁶, or 10⁷ exogenous (e.g., human) platelets/ ml blood 6 weeks post-administration into the immunocompromised mouse (e g., 7, 8, 9, or 10 weeks post-administration).

In some embodiments, the media comprises 1-100 nM TPO or TPO analog (e.g., 1-50, 1-20, 5-15, or about 10 nM TPO or TPO analog). In some embodiments, the media comprises 1-100 ng/ml IL-1 (e.g., 1-50, 1-20, 5-15, or about 10 ng/ml IL-1). In some embodiments, the IL-1 is IL-1a (e.g., recombinant human IL-1α). In some embodiments, the media comprises 5-15 ng/ml IL-1α and 5-15 nM TPO or TPO analog. In some embodiments, the media lacks serum and other animal products (e.g., serum is not added to the media or is not detectable in the media). In some embodiments, the conditions lack feeder cells (e.g., feeder cells are not added or are undetectable).

In some embodiments, the population of cells comprise at least 30, 40, 50, 60, 70, 80, 90 or higher percentage of CD34+CD41+ MKPs. In some embodiments, at least 50% of the CD34+CD41+ MKPs are CD184+ (e.g., 55, 60, 70, 75, 80, 85% or higher percentage CD184+).

In some embodiments, less than 75% of the CD34+CD41+ MKPs are CD42a+ (e.g., less than 60, 55, 50, 45, 40, 35, 30, 25, 20, 10, 5% or lower percentage of CD42a+). In some embodiments, the population of cells lacks B and T cells (e.g., less than 5, 4, 3, 2, or 1% B and T cells, or undetectable levels of B and T cells). In some embodiments, the method further comprises separating the MKPs from the population of cells. In some embodiments, CD34+CD41+ MKPs are separated. In some embodiments, CD34+CD41+CD184+ MKPs are separated. In some embodiments, CD34+CD41+CD42a^{neg} MKPs are separated.

In some embodiments, the HSCs are cryopreserved prior to culturing. In some embodiments, the method further comprises cryopreserving the population of cells. In some embodiments, the cells retain function post-cryopreservation, e.g., platelet generating activity, as describe herein. In some embodiments, the population of cells retains at least 60, 70, 75, 80, 85, 90, 95, 100% or higher percent function post-cryopreservation compared to the population of cells pre-cryopreservation.

In some embodiments, the method further comprises allowing the MKPs to generate platelets. In some embodiments, the allowing is carried out in vitro in Media comprising SCF or an SCF analog; TPO or a TPO analog; and heparin. In some embodiments, the allowing comprises administering said MKPs to a recipient to generate platelets in vivo.

Further provided is a population of cells generated according to the methods described herein, i.e., culturing CD34+ hematopoietic stem cells (HSCs) in media comprising thrombopoietin (TPO) or a TPO analog and IL1-α under conditions that permit the CD34+ HSCs to generate a population of cells comprising CD34+CD41+ MKPs, wherein the MKPs have platelet generating activity.

In addition, provided herein are methods for generating platelets in an individual, e.g., a thrombopoietic individual. In some embodiments, the method comprises culturing CD34+ hematopoietic stem cells (HSCs) in media comprising thrombopoietin (TPO) or a TPO analog and IL1-α under conditions that permit the CD34+ FISCs to generate a population of cells comprising CD34+CD41+ megakaryocyte progenitor cells (MKPs) with platelet generating activity, and administering the MKPs to an individual, thereby generating platelets in the individual. In some embodiments, at least 0.2×10⁵ cells from the population of cells per kg body weight are administered to the individual (e.g., at least 0.5×10⁵., 0.75×10⁵, 10⁵, 0.5×10⁶, 10⁶ , 5×10⁶ cells/kg body weight).

In some embodiments, the HSCs are human. In some embodiments, the HSCs are obtained from a plurality of individuals. In some embodiments, the HSCs (and MKPs) have a mismatch at one or more MHC (or HLA) loci. In some embodiments, the HSCs (and MKPs) have a mismatch at one or more MHC (or HLA) loci with the recipient (i.e., the individual to which the MKPs are administered). In some embodiments, the population of cells is characterized in that administration of 5×10⁶ cells from the population of cells into an immunocompromised mouse results in at least 10⁶, e.g., at least any one of 5×10⁶, 10⁷, 5×10⁷, or more human platelets/ml blood 14 days post-administration.

In some embodiments, the HSCs are cryopreserved prior to culture. In some embodiments, the population of cells is cryopreserved prior to administration. In some embodiments, the method further comprises separating the MKPs from the population of cells prior to administering. In some embodiments, CD34+CD41+ MKPs are separated. In some embodiments, CD34+CD41+CD184+ MKPs arc separated. In some embodiments, CD34+CD41+CD42a^{neg} MKPs are separated. In some embodiments, the MKPs are cryopreserved prior to administering.

### BRIEF DESCRIPTION OF THE DRAWINGS

FIG. 1 shows a schematic of hematopoietic lineage cells with a focus on the megakaryocyte line.

FIG. 2 shows MKP populations generated according to the present methods. (A) Cells expand more than 15-fold at day 8 in culture. (B) MKPs make up more than 50% of the cells in culture. (C) The MKPs generated according to the present methods are primarily CD184+ (CXCR4), and are better able to engraft and generate more platelets in vivo than CD184^{neg} MKPs.

FIG. 3 compares the MKPs generated as described herein (labeled CLT-009) with myeloid progenitor cells expanded under different conditions (labeled CLT-008, see, e.g., WO2007095594). The MKPs are more committed to the megakaryocyte lineage, as shown in the upper panels with MK promoting conditions. In addition, the MKPs are less capable of forming neutrophils under neutrophil promoting conditions.

FIG. 4 (A) Representative cell surface staining pattern of the present MKPs at day 8 of culture. The cells are distinguishable from CD34+ HSCs as CD41+ and CD90^{neg}. (left panels). The right panels show the high level of CD184+ expression on the present CD41+ MKPs. (B) Results from Megacult assay show that the present MKPs (T1a=TPO mimetic+IL-1 alpha) generate significantly more megakaryocyte colonies that the myeloid progenitor cells (labeled CLT-008).

FIG. 5 shows fold expansion of cells from 3 different human donors (and pooled) at day 8 in culture. The bottom panel shows the relative populations of cells over culture days 0-16. The number of MKPs (CD34+CD41+) is maximized at about day 8. Prior to that time, the culture includes a higher percentage of the starting population (HSCs), while after that time, the MKPs differentiate into more mature megakaryocytes (CD34^{neg}CD41+CD42a+).

FIG. 6 shows that the present MKPs are capable of megakaryocyte colony formation before and after cryopreservation. The top panel A shows the results of methyl-cellulose culture that promotes general myeloid cell development. The bottom panel B shows the results of megacult conditions that promote megakaryocyte development. Cryopreservation reduced megakaryocyte CFU (colony forming units) and BFU (blast forming units) only slightly. GM=granulocyte-macrophage; M and MK=megakaryocyte; G=granulocyte; CFU-E=erythrocyte.

FIG. 7 shows the timeline of engraftment in the indicated tissues of xenograft mice after administration with 10⁷ day 8, post-cryopreserved, human MKPs.

FIG. 8 shows that the present MKPs (post-cryopreservation) can provide rapid replacement of platelets in xenograft mice (human platelets—middle line). Human platelet number increase immediately after administration, with numbers peaking at around 2-3 weeks (˜3×10⁴ platelets/ul blood). In the case of thrombocytopenia, this rapid increase in platelet numbers can provide time for endogenous platelet production to recover.

FIG. 9 shows that day 8 culture, post-cryopreservation, includes a higher percentage of MKPs (CD34+CD41+) than post-cryopreserved day 10 culture. Day 10 culture, however, includes a higher percentage of late stage MKPs (CD41+CD42a+).

FIG. 10 compares the in vivo platelet production of the post-cryopreserved day 8 and day 10 MKPs shown in FIG. 10. Administration of 10⁷ day 8 MKPs produce more platelets more rapidly than 10⁷ day 10 MKPs. Platelet numbers from the day 8 MKPs peaked at about 2 weeks (about 2×10⁴/ul blood), while platelet numbers from day 10 MKPs peaked at about 3 weeks (about 3×10³/ul blood).

FIG. 11 shows that human platelets generated in vivo (in mice) from the present MKPs are functional. (A) Platelets isolated from human blood show surface expression of CD62P upon exposure to ADP (adenosine diphosphate). (B) Similarly, human MKP-generated platelets obtained from xenotransplant mice show surface expression of CD62P upon exposure to ADP.

FIG. 12 shows that human platelets derived from MKPs generated as described herein release platelet factor 4 upon activation, demonstrating that the platelets are functional.

FIG. 13 shows the total fold expansion of CD34+ cells isolated from three individual donors cultured in various conditions. T1a indicates TPO mimetic+IL-1a. T1a9 indicates TPO mimetic+IL-1a+IL-9. The composition of the expanded cells after eight days in culture is shown with regard to CD34, CD41 and CD42a expression. The data show that IL-9 increases the size of all populations, including the potent CD34+CD41+ populations.

FIG. 14 shows the growth over eight days of representative MKP expansion cultures from a pool of CD34+ donors using 10 nM TPO mimetic, 10 ng/ml IL-1a and 60 ng/l IL-9. 1% human serum albumin (HSA) was added as indicated. The cultures were carried out in either static bag cultures or in agitated shaker flasks as indicated. The data demonstrate that addition of HSA leads to higher fold expansion. The data also show that MKP cultured cells are amenable to agitation, which facilitates scaling up the cultures for production.

FIG. 15 shows the phenotypic analysis of the cultures presented in FIG. 14 after eight days of culture. (A) shows that addition of HSA leads to slightly reduced relative numbers of the potent CD34+CD41+ cells. (B) shows that the relative decrease in CD34+CD41+ cells is more than compensated by the total increase in cells, resulting in a higher total yield of CD34+CD41+ cells. In addition, the number of CD34+CD41+ cells is maintained or increased when cultured in agitated shaker flasks.

FIG. 16 shows the average fold expansion of three individual MKP cultures over eight days when cultured in shaker flasks in the presence of 10 nM TPO mimetic, 10 ng/ml IL-1a, 60 ng/ml IL-9 with or without additional 1% HSA. Cell numbers increased by nearly 50% in the presence of HSA (A), and the cellular composition of the expanded MKPs was maintained (B).

### DETAILED DESCRIPTION OF THE INVENTION

### I. Introduction

Provided herein are megakaryocyte progenitor cells (MKPs) that rapidly produce significant numbers of platelets when administered in vivo. The increase in circulating platelets persists in the recipient for several weeks. Further provided are methods and compositions for generating the high platelet-producing megakaryocyte lineage cells from hematopoietic stem cells (HSCs) in the absence of feeder cells and serum. The megakaryocyte progenitor cells (MKPs) described herein can be used, among other things, to treat thrombocytopenia and related conditions.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

The term “megakaryocyte progenitor cell” or “MKP” refers to a progenitor cell that is predominantly committed to the megakaryocyte lineage. MKPs are described in more detail below, but are typically characterized by surface expression of CD41. MKPs can be isolated from the bone marrow (or from mobilized peripheral blood) by selecting for CD34+CD41+ cells. Early MKPs show little to no CD42a surface expression, while later stage MKPs express CD42a on the surface.

The term “platelet generating activity” means that a population of cells is capable of producing platelets either in vivo or in vitro as described herein. Platelets are directly produced by mature megakaryocytes, but mature megakaryocytes are not capable of dividing due to their polyploidy. MKPs are thus said to have platelet generating activity if they are capable of giving rise to platelet producing megakaryocytes.

The term “immunocompromised” generally refers to an individual that has been genetically modified or physically treated (e.g., irradiated, infected) in such a way to reduce immune function in the individual (innate or adaptive immunity, or both). For example, functional B and T cells can be largely eliminated by compromising DNA recombination, either genetically (e.g., by knocking out genes required for recombination) or with ionizing radiation.

Immune cells, upon immunogenic challenge, often start dividing, making them vulnerable to any number of agents, such as ionizing radiation and DNA interchelators. Certain viruses, fungi, and bacteria preferentially target cells of the immune system (e.g., HIV). Typically, an “immunocompromised mouse” refers to genetic models of immunodeficiency, e.g., SCID, RAG deficient, NSG (NOD/SCID/Gamma, e.g., NOD.Cg-Prkdc^{scid} I12rg^{tmlWjl}/SzJ), Nude, etc.

The term “analog” as used herein encompasses mimetics (peptide or small molecule mimetics), recombinantly produced forms, functional fragments, and variants (e.g , species homologs, allelic variants) of a given factor. The term “thrombopoietin analog” (TPO analog) thus encompasses TPO mimetics, recombinant TPO (e.g., rhTPO), functional fragments of TPO, species homologs, and allelic variants that bind to the thrombopoietin receptor.

The terms “therapy,” “treatment,” and “amelioration” refer to any reduction in the severity of symptoms, or improvement in therapeutic parameters (e.g., platelet numbers, clotting ability, etc.). As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (normal platelet production restored) or partial, such that more platelets are found in a patient than would have occurred without the present invention. The effect of treatment can be compared to an individual or pool of individuals not receiving the treatment, or to the same patient prior to treatment or at a different time during treatment. In some aspects, the severity of the condition (e.g., thrombocytopenia) is reduced by at least 10%, as compared, e.g., to the individual before administration or to a control individual not undergoing treatment. In some aspects the severity of condition is reduced by at least 25%, 50%, 75%, 80%, or 90%, or in some cases, no longer detectable using standard diagnostic techniques (i.e., platelet numbers are within a normal range).

The terms “transplant”, “transfusion”, “administration”, and “injection,” when referring to cells, are largely synonymous as used herein, and refer to administration of cells to an individual. The transplant can be allogeneic or autologous, or as described in the examples, a xenograft.

The terms “effective amount,” “effective dose,” “therapeutically effective amount,” etc. refer to that amount of the therapeutic agent sufficient to ameliorate a disorder, e.g., thrombocytopenia. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose can refer to the total number of cells, number of cells per kg body weight, or concentration of cells. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc. A patient can include individuals that have not received treatment, are currently receiving treatment, have had surgery, and those that have discontinued treatment.

A “cell culture” is an in vitro population of cells residing outside of an organism. The cell culture can be established from primary cells isolated from a cell bank or animal, or secondary cells that are derived from one of these sources and immortalized for long-term in vitro cultures.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. The term does not imply that all cells in the culture survive or grow or divide, as some may naturally senesce, etc. Cells are typically cultured in media, which can be changed during the course of the culture.

The terms “media” and “culture solution” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/ or differentiation such that the original cell is no longer exists, but the continuing cells will be understood to derive from the same source.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

“Allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

“Autologous” refers to deriving from or originating in the same subject or patient. An “autologous transplant” refers to collection and retransplant of a subject's own cells or organs.

“Graft-versus-host response” or “GVH” or “GVHD” refers to a cellular response that occurs when lymphocytes of a different MHC (major histocompatibility complex) class are introduced into a host, resulting in the reaction of the lymphocytes against the host. The HLA (human leukocyte antigens) are a subset of antigen presenting MHC proteins found in humans (see, e.g., Bodner et al. (1992) Human Immunol. 34:4).

### III. Cells

A “hematopoietic stem cell” or “HSC” refers to a clonogenic, self-renewing pluripotent cell capable of ultimately differentiating into all cell types of the hematopoietic system, including

B cells T cells, NK cells, lymphoid dendritic cells, myeloid cells, granulocytes, macrophages, megakaryocytes, and erythroid cells. As with other cells of the hematopoietic system, HSCs are typically defined by the presence of characteristic cell surface markers. For example, in humans, HSCs can be identified by cell surface expression of CD34, CD90 (Thy 1), CD 117 (c-Kit), CD59, and CD150. Human HSCs typically do not express CD38. Murine HSCs can be identified by cell surface expression of Seal, CD117 (c-Kit), and Thy1.1, and low or undetectable expression of CD34.

HSCs can be obtained from the bone marrow (BM), placenta, embryonic tissue, umbilical cord blood (UCB), or from peripheral blood (PB). HSCs can be mobilized from the bone marrow to the peripheral blood of an individual, e.g., by administering G-CSF (see e.g., Alexander et al. (2011) Transfusion; Roncon et al. (2010) Transplant Proc 43:244). Where applicable, stem cells can be mobilized from the bone marrow into the peripheral blood by prior administration of cytokines or drugs to the subject (see, e.g., Lapidot et al., Exp. Hematol. 30:973-981 (2002)). Cytokines and chemokines capable of inducing mobilization include granulocyte colony stimulating factor (G-CSF), granulocyte macrophage colony stimulating factor (GM-CSF), erythropoietin (Kiessinger et al., Exp. Hematol. 23:609-612 (1995)), stem cell factor (SCF), AMD3100 (AnorMed, Vancouver, Canada), interleukin-8 (IL-8), and variants of these factors (e.g., pegfilgastrim, darbopoietin). Combinations of cytokines and/or chemokines, such as G-CSF and SCF or GM-CSF and G-CSF, can act synergistically to promote mobilization and may be used to increase the number of HSCs and progenitor cells in the peripheral blood, e.g., for subjects who do not show efficient mobilization with a single cytokine or chemokine (Morris et al., J. Haematol. 120:413-423 (2003)). G-CSF is available commercially, e.g., as filgrastim; lenograstim; pluripoietin, Neupogen™, granulokine (Amgen, Calif., USA), and granocyte (Rhone-Poulenc). GM-CSF is also available commercially, e.g., as molgramostin and sargramostim. In addition, HSCs can be obtained from induced pluripotent stem cells (iPS), which are generated from more differentiated cells by ectopic expression of reprogramming factors, e.g., OCT4, SOX2, KLF4, cMYC, LIN28, and/or NANOG (see, e.g., Kamata et al. (2010) Hum. Gene Ther. 21:1555; Takahashi et al. (2007) Cell 131:861; Amabile & Meissner (2009) Trends Mol Med 15:59).

Myeloid progenitor cells can comprise one or more of: common myeloid progenitor cells (CMP); and the committed myeloid progenitors: erythroid/megakaryocytic progenitor (MEP), granulocyte/monocyte progenitors (GMP); and megakaryocyte progenitor (MKP).

Common myeloid progenitor (CMP) cells are a hematopoietic progenitor subset that can give rise to all lineages of myeloerythroid cells, but not lymphoid lineages. CMP cells can be identified and isolated by means of cell surface markers. Both human and murine CMP cells stain negatively for the markers Thy-1 (CD90), IL-7R-alpha (CD127); and lineage markers that include CD2; CD3; CD4; CD7; CD8; CD10; CD11b; CD14; CD19; CD20; CD56; and glycophorin A (GPA) in humans and CD2; CD3; CD4; CD8; CD19; IgM; Ter110; and Gr-1 in mice. CMP cells are CD34+CD38+. In humans, CMP cells are also characterized as IL-3R-alpha^{lo} CD45RA^{neg}. Mouse CMP cells are Sea-1 negative, (Ly-6E and Ly-6A), c-kit high, and Fc-gamma-R^{low}.

Megakaryocyte/erythroid progenitor cells (MEP), which derive from CMPs, are characterized by the ability to differentiate into committed megakaryocyte progenitor and erythroid progenitor cells. Mature megakaryocytes are described below in more detail. Erythroid cells are formed from the committed erythroid progenitor cells through a process regulated by erythropoietin, and ultimately differentiate into mature red blood cells. Murine MEPs can be characterized by cell marker phenotype c-Kit^{high}, IL-7R^{neg}, FcR^{low} and/or CD34^{low}. Murine MEP cell populations can also be characterized by the absence of markers B220, Ter1119, CD4, CD8, CD3, Gr-1, and CD90. Human MEPs can be identified by cell surface markers CD34+ CD38+ CD123^{neg} CD45RA^{neg}. Human MEP cell populations can also be characterized by the absence of markers CD3, CD4, CD7, CD8, CD10, CD11b, CD14, CD19, CD20, CD56, and CD235a.

Further restricted progenitor cells in the myeloid lineage are the granulocyte progenitor, macrophage progenitor, megakaryocyte progenitor, and erythroid progenitor.

Megakaryocyte progenitor (MKP) cells are hematopoietic progenitors restricted to the megakaryocytic lineage. MKP cells express detectable levels of the markers CD41 and CD34. MKPs can be further selected for a lack of expression of the markers Thy-1 (CD90), IL-7R-alpha (CD127); and/or with a lineage panel of markers. Human MKP cells are typically negative for CD2, CD3, CD7, CD8, CD10, CD11b, CD14, CD19, CD20, CD56, CD45L, CD71, CD235a, and GPA, and positive for CD33, CD38, CD184, and c-Kit (CD 117). Expression of CD42a and CD9 increase with maturation, and can be associated with more mature megakaryocytes and platelets. Expression of CD34 decreases with maturation. Mouse MKP cells are typically negative for CD2; CD3; CD4; CD8; CD19; IgM; Ter119; and Gr-1.

The developmental potential of progenitor cells can be validated by in vitro culture. For example, MKPs can be cultured as described herein to give rise to megakaryocytes (CFU-MK) and platelets. CMPs can be cultured in the presence of steel factor (SCF), flt-3 ligand (FL). interleukin (IL)-3, IL-11, GM-CSF, thrombopoietin (TPO) and/or erythropoietin (EPO). CMP cells will give rise to myeloerythroid colonies including CFU-GEMMeg (mixed myeloid, megakaryoid, and erythroid lineage), burst-forming unit-erythroid (BFU-E), CFU-megakaryocytes (CFU-Meg), CFU-granulocyte/macrophage (CFU-GM), CFU-granulocyte (CFU-G) and CFU-macrophage (CFU-M).

As MKPs mature into megakaryocytes, the cells become polyploid and no longer capable of dividing. The cytoplasm expands, and a network of specialized membranes forms within the cytoplasm, i.e., the demarcation membranes system (DMS). These changes support production of large numbers of platelets. Mature megakaryocytes are characterized by proplatelet extensions from the cell, and release of platelets (thrombocytopoiesis). A megakaryocyte typically releases 2000-12,000 platelets. See Reems et al. (2010) Transfusion Med. Rev. 24:33 for a review.

Platelets lack nuclei, and have a limited life span of less than about 10 days in vivo. Platelets retain many of the cell surface markers of the megakaryocyte lineage, e.g., CD41 and CD42a. Functional markers, e.g., involved in blood clotting, include receptors for von Willebrand factor (GPIb-V-IX), fibrinogen (GPIIb/IIIa), and collagen (GPVI) (Nishikii et al. (2008) J. Exp. Med. 4:1917; Kehrel et al. (1998) Blood 91:491). Expression of CD62p (P-selectin) indicates platelet activation.

### IV. Methods of Generating MKPs

Provided herein are methods for generating megakaryocyte lineage cells from CD34+ HSCs. Methods for identifying and obtaining HSCs are described herein. HSCs to be used for generating MKPs and/or megakaryocytes can be obtained from a single donor, e.g., for autologous or allogeneic administration to a recipient. The donor can be related to the recipient or unrelated. The HSCs can also be obtained from a plurality of individuals. For example, in some embodiments, the HSCs are from a pooled source, e.g., from a cord blood bank. HSCs can be obtained from humans, other primates, livestock (sheep, cows, horses, pigs, etc.), companion animals (dogs, cats, rabbits, etc.), mice, or rats.

Surprisingly, the HSCs described herein can be frozen (cryopreserved) prior to differentiation. Methods for freezing and thawing cells while maintaining viability are known in the art (Phelan (1998) Curr. Prot. Cell Biol 1.1.1). Typically, cells are suspended at a density of about 10⁶-10⁸/mL in media comprising DMSO (e.g., 2-10% DMSO), and quickly frozen at about −70 to −100° C. In some embodiments, the cryopreservation lacks animal products such as serum or other animal proteins. In some cases, the cryopreservation media includes serum (e.g., FCS) or other proteins (e.g., recombinant proteins). Thawing is typically carried out gradually, by adding media to the cells until the sample is brought up to 37° C.

In some embodiments, the CD34+ HSC is from a human. In some embodiments, the CD34+ HSC is from a cryopreserved sample, e.g., from a cryopreserved sample of cells comprising CD34+ HSCs. In some embodiments, the cryopreserved sample represents a pool of cells obtained from more than one donor.

Methods for generating an MKP can comprise culturing a population of CD34+ HSCs in media comprising thrombopoietin (TPO), or a TPO analog, and IL-1 for a time sufficient to generate an MKP.

In some embodiments, the IL-1 is IL-1 alpha. In some embodiments, the media comprises 1-100 ng/ml IL-1, e.g., 1-50 ng/ml IL-1, 5-20 ng/ml IL-1, 5-15 ng/ml IL-1, 8-12 ng/ml IL-1, or about 10 ng/ml IL-1. In some embodiments, the TPO analog is AF15705. In some embodiments, the media comprises an amount of TPO or TPO analog that is functionally equivalent to 1-100 nM AF15705, e.g., 1-50 nM, 5-20 nM, 5-15 nM, 8-12 nM, or about 10 nM. One of skill will recognize that various forms of TPO and TPO analogs have different molecular weights, so that the functionally equivalent amounts may differ, e.g., if expressed in terms of ng or ug/ml. In the present context, functional equivalence is determined by the number of thrombopoietin receptor binding units in the solution of TPO, TPO variant, or TPO analog.

In some embodiments, the media further comprises at least one additional factor selected from: SCF, IL-6, IL-9, IL-11, and IL-20. Thus, in some embodiments, the media further comprises SCF or an analog thereof. In some embodiments, the media further comprises IL-3 or an analog thereof. In some embodiments, the media is devoid of IL-3 (i.e., IL-3 is not added to the media, and the media lacks detectable IL-3). In some embodiments, the media further comprises IL-6 or an analog thereof. In some embodiments, the media further comprises IL-9 or an analog thereof. In some embodiments, the media further comprises IL-11 or an analog thereof. In some embodiments, the media further comprises IL-20 or an analog thereof. More than one of the additional factors can be added in any combination (e.g., TPO+IL-1+SCF+IL-9; TPO+IL-1+SCF+IL-6, etc.).

In some embodiments, the media does not include serum or feeder cells, i.e., serum and feeder cells are not added to the media, and the components are not present at a significant level in the media. In some embodiments, the media lacks detectable levels of serum and feeder cells. In some embodiments, the culturing is carried out for at least 3, 5, 6, 7, or 8 days. In some embodiments, the culturing is carried out for 5-12, 5-15, 8-15, or 6-10 days. In some embodiments, the culturing is carried out for 6, 7, 8, 9, 10, 12, or 15 days.

In some embodiments, the culturing results in at least a 5-fold increase in total nucleated cell (TNC) numbers, e.g., after a 5, 6, 7, 8, 9, or 10 day culture period. In some embodiments, the culturing results in a 5-10 or 5-15 fold increase in TNC numbers, e.g., 6, 7, 8, 9, 10, 11, 12, 13, 14, 15, or higher fold increase in TNC numbers over the culture period.

In some embodiments, the MKP is CD41+ CD42a^{neg} (early stage MKP). In some embodiments, the MKP is CD41+CD42a+ (late stage MKP). In some embodiments, the method generates a plurality of MKPs comprising CD41+CD42a+ MKPs and CD41±CD42a^{neg} MKPs. In some embodiments, the culture includes at least 50% MKPs, e.g., at least 60, 70, 80, 90, or higher percentage MKPs. In some embodiments, the cells are further separated to produce a relatively pure population of MKPs, e.g., using FACS sorting, antibody-coated beads or other affinity methods. In this case, the MKPs can be separated using positive selection for CD34 and CD41, and optionally CD42a and CD184. In some embodiments, the MKPs are positively selected for expression of CD34, CD41, and CD184. In some embodiments, the CD34+CD41+ MKPs are CD184+ (e.g., greater than 50% CD184+, e.g., about 50-100% or about 70-100% CD184+).

General cell culture practices are known, and can be applied by one of skill in the art as appropriate (see, e.g., Freshney, Culture of Animal Cells: A manual of basic technique and specialized applications (6^{th} ed. 2010)). Cells are typically grown in conditions that are close to physiological conditions, e.g., around 37° C., in 5% CO₂, with controlled humidity. The culture is sterile to avoid bacterial, fungal, or other contamination. The cells can be grown in culture bags, flasks, plates, or multi-well plates, depending, e.g., on the scale of the culture. Culture bags are generally gas permeable and available in various sizes. Commercial vendors include Miltenyi Biotech and Origen BioMedical Inc.

In some embodiments, the method further comprises freezing (freeze drying, cryopreserving, etc.) the MKPs. As noted above, methods for freezing and thawing cells are known in the art, and reagents are commercially available (see, e.g., Materials and Methods, and Phelan (1998) Curr. Prot. Cell Biol 1.1.1). In some embodiments, the cryopreservation media lacks animal products (e.g., serum). In some embodiments, the MKPs can be frozen in a dosage form, e.g., so that each frozen sample comprises a therapeutically effective amount of MKPs capable of generating platelets when administered to an individual (see Therapeutic application section below).

The MKPs generated using the methods described here can further differentiate into megakaryocytes (CD34^{neg}CD41+CD42a+) and rapidly produce large numbers platelets both in vitro and in vivo. In some embodiments, when 5×10⁶ of the MKPs are administered to an immunocompromised mouse (such as an irradiated or genetically deficient mouse, e.g., NOD, SCID, NSG, nude, etc.), the MKP cells will generate at least 10⁶ exogenous platelets/ml blood 14 days post-administration (e.g., at least 2×10⁶, 5×10⁶, 10⁷ , 2×10⁷, 5×10⁷, or 10⁸ exogenous platelets/ ml blood). In some embodiments, administration of 10⁷ MKPs into an immunocompromised mouse results in at least 10⁶ exogenous platelets/ ml blood 14 days post-administration (e.g., at least 2×10⁶, 5×10⁶, 10⁷ , 2×10⁷, 5×10⁷, or 10⁸ exogenous platelets/ml blood). In some embodiments, administration of 10⁷ MKPs into an immunocompromised mouse results in at least 10⁶ (e.g., 0.2, 0.4, 0.5, 0.75, or 1×10⁷) exogenous platelets/ml blood 6 weeks post-administration. In some embodiments, administration of 10⁷ MKPs into an immunocompromised mouse results in at least 10⁶ (e.g., 0.2, 0.4, 0.5, 0.75, or 1×10⁷) exogenous platelets/ml blood 8 weeks post-administration.

In some embodiments, the method comprises culturing a CD34+ HSC in media comprising about 10 ng/ml IL-1 alpha (e.g., 5-15 ng/ml, 8-12 ng/ml, etc.) and about 10 nM TPO or TPO analog (e.g., an amount of TPO or TPO analog that is functionally equivalent to about 10 nM AF15705, e.g., 5-15 nM, 8-12 nM, etc.) for at least 5 days, e.g., 7, 8, or 9 days, thereby generating an MKP. In some embodiments, the culturing is in a culture bag. In some embodiments, the culture is static (e.g., not agitated or shaken). In some embodiments, the method further comprises cryopreserving the MKP.

The MKPs and/or megakaryocytes described herein do not require MHC matching between the donor(s) and recipient. Thus, administration of the cells typically does not result in GVHD, regardless of MHC mismatching. “Graft versus host response,” “Graft versus host disease,” or “GVHR” or “GVHD,” refers to the immune response that occurs when lymphocytes of a different MHC class are introduced into a host, resulting in the reaction of the lymphocytes against the host.

The MHC (major histocompatibility complex) is a protein complex involved in antigen presentation on the cell surface. All nucleated cells express MHCI, while MHCII is expressed by a subset of immune cells. There are several polymorphic versions of the MHC genes, which result in the GVHD referenced above. The most commonly studied MHC genes are those from the HLA (human leukocyte antigen), which include HLA-A, HLA-B, HLA-C, HLA-DPA1, HLA-DPA2, HLA-DQA1, HLA-DQA2, HLA-DRA1, HLA-DRB1, HLA-DRB3, HLA-DRB4 and HLA-DRBS. Genes within each of these groups are highly polymorphic, as reflected in the numerous HLA alleles or variants found in the human population, and differences in these groups between individuals is associated with the strength of the immune response against transplanted cells. Even within a single family, the variety of combinations can lead to complications when a perfect match is required between a cell donor and recipient.

In some embodiments, the MKPs and/or megakaryocytes include at least one mismatch at the MHC, either within the population of cells for administration (donor cells), or between the donor cells and the recipient. In some cases, the MKPs and/or megakaryocytes are derived from HSCs obtained from a plurality of allogeneic donors, so that the MKPs and/or megakaryocytes are mismatched within the sample, and mismatched with any given recipient. In some embodiments, the MKPs and/or megakaryocytes are at least partially mismatched at one MHC locus. In some embodiments, the MKPs and/or megakaryocytes represent a mixed population, e.g., are derived from a mixture of HSCs, obtained from more than one individual. Where the MKPs and/or megakaryocytes are from or derived from cells obtained from more than one donor, they are said to be a “mixture” or “derived from a mixture” of cells.

In some embodiments, the MKPs and/or megakaryocytes are derived from cells obtained from a single donor (or from a clonogenic cell line) such that the population of cells is matched at the MHC loci. These matched MKPs and/or megakaryocytes can be administered to an individual that is either matched or mismatched (e.g., mismatched for at least one MHC gene, partially mismatched, or completely mismatched) at the MHC loci. In some embodiments, the MKPs and/or megakaryocytes are derived from cells obtained from the eventual recipient of the cells (e.g., for autologous transplant), or from an individual with identical MHC genes, so that the donor cells are matched at the MHC loci with the recipient.

Determining the degree of MHC mismatch can rely on standard tests known and used in the art. The sequences of the human and mouse MHC genes are known in the art and available from Genbank (e.g., found at the NCBI website). Molecular methods for determining MHC type generally employ probes and/or primers to detect specific gene sequences that encode the HLA protein. Specific oligonucleotides can used as hybridization probes to detect restriction fragment length polymorphisms (RFLPs) associated with particular HLA types (Vaughn, Methods in Molecular Biology: MHC Protocols 210:45-60 (2002)). Primers can be used for amplifying HLA sequences (e.g., by polymerase chain reaction or ligation chain reaction). The primers can be designed to be specific for particular HLA sequences, or the PCR products can be further examined by direct DNA sequencing, restriction fragment polymorphism analysis (RFLP), or hybridization with a series of sequence specific oligonucleotide primers (SSOP) (Petersdorf, et al., Blood 92:3515-20 (1998); Morishima et al., Blood 99:4200-6 (2002); and Middleton and Williams, Methods in Molecular Biology: MHC Protocols 210:67-112 (2002)).

In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (Mickelson and Petersdorf, Hematopoietic Cell Transplantation, Thomas et al. eds., pg 28-37, Blackwell Scientific, Malden, Mass. (1999). Other cell-based assays include flow cytometry using labeled antibodies or enzyme linked immunoassays (ELISA).

The initial population of cells (HSCs) is cultured ex vivo by contacting the cells with a medium having a cytokine and growth factor mixture permissive for expansion of megakaryocyte lineage cells. Cytokines are proteins made by cells that modulate the physiological state of cells, whether the cell is another cell or the cell producing the cytokine. Cytokines made by lymphocytes are often described as lymphokines (IL). Growth factors are also compounds made by cells that affect the proliferation and differentiation of cells, whether the cell is another cell or the cell producing the growth factor. Cytokines and growth factors generally act on cells via receptors.

For the methods described herein, cytokines and growth factors are chosen to expand populations of the megakaryocyte lineage, e.g., megakaryocyte progenitor cells (MKPs). As the cells differentiate and commit to the megakaryocyte lineage, they have limited or no self-renewing capacity. The culture conditions are selected to support division of cells that develop into megakaryocytes, while limiting or minimizing growth and expansion of other cell types.

Cytokines and growth factors for generating MKPs and/or megakaryocytes are generally selected from TPO, IL-1 (IL-1 alpha, IL-1 beta), SCF, IL-3, IL-6, IL-9, IL-11, IL-20, and analogs thereof. The cytokines can be naturally occurring products, recombinant products, variants, or modified forms having similar biological activity as the naturally occurring forms such as, e.g., peptide mimetics. The cytokines can also be selected from fusion proteins or engineered cytokines (e.g., Curtis et al. Proc. Natl. Acad. Sci. U.S.A. 1991 88:5809-5813; Lu et al. Exp. Hematol. 1995 23, 1130-1134; Zhao et al. Stem Cells 1994, 12:1130-1134). Typically, the growth factors and cytokines in the culture will be derived from the same species as the cells in the culture. That is, for culturing human HSCs, the cytokines and/or growth factors will be derived from human sequences (e.g., if recombinantly produced), and/or will functionally interact with the human cells (e.g., for synthetic analogs).

Thrombopoietin (TPO), in its naturally occurring form, is a glycosylated peptide, though functional recombinant forms and analogs are known. TPO exerts its effects via binding to the proto-oncogene c-mpl, and can stimulate the differentiation of stem cells into megakaryocyte progenitor cells, induce the expression of megakaryocyte differentiation markers, promote megakaryocyte proliferation and polyploidization, and increase the number of platelets in circulation (see, e.g., Lok et al., Stem Cells 12:586-98 (1994)). TPO is also known as megakaryocyte growth and differentiation factor (MGDF) or c-Mpl ligand. Amino acid and nucleic acid sequences for thrombopoietin are known and publically available. Recombinant TPO, e.g., rhTPO, has been used in clinical trials. Recombinant and variant forms of TPO are described e.g., in Souryi et al., Cell 63:1137-1147 (1990); Gurney et al., Blood 85:981-8 (1995); Wada et al., Biochem Biophys Res Commun. 213:1091-8 (1995); Park et al., J Biol Chem. 273:256-61 (1998); and Jagerschmidt et al., Biochem. J. 333 (Pt 3):729-34 (1998).

TPO analogs (mimetics) include AF15705 (non-PEGylated) and GW395058 (PEGylated) (see, e.g., de Serres et al. (1999) Stem Cells 17:203). Additional TPO analogs include the Nplate® “peptibody” (romiplostim, Amgen, Thousand Oaks, Calif.), eltrombopag (GlaxoSmithKline, Philadelphia, Pa.), and the mimetics described in U.S. Pat. No. 7,674,887.

TPO and TPO analogs can be administered according to protocols as known in the art. For example, rhTPO can be administered intravenously by bolus injection at doses ranging from 0.1 to 10 μg/kg/day every one to 3 days. G-CSF can also be administered to promote myeloid recovery (see, e.g., Bone Marrow Transplantation (2001) 27:261-268).

SCF, also known as c-Kit ligand, mast cell growth factor, or Steel factor, acts on multiple levels of the hematopoietic hierarchy to promote cell survival, proliferation, differentiation, adhesion and functional activation in combination with other cytokines. SCF can act on myeloid cells (e.g., mast cells), multipotent stem and progenitor cells, megakaryocytes, and a subset of lymphoid progenitors (Broudy , Blood 90(4):1345-1364 (1997)). SCF exerts its biological effects by binding to its receptor, C-KIT. Naturally occurring SCF is synthesized by bone marrow stromal cells as either a transmembrane form or a soluble form, both of which are biologically active. Amino acid and nucleic acid sequences are known for SCF and are publically available. These include, e.g., murine (Lyman, et al., Cell 75:1157-67 (1993)), rat (Martin et al., Cell 63:203-11 (1990)); and human (Martin, et al.). Recombinant SCF and variants are described in Jones et al., J. Biol. Chem. 271:11301 (1996); Lu et al., J. Biol. Chem. 271:11309 (1996); Langley et al., Arch. Biochem. Biophys. 295:21 (1992); Lev et al., Mol Cell Biol. 13(4):2224-34 (1993); and Langley et al., Arch. Biochem. Biophys. 311:55-61 (1994).

IL-1 is involved in the up- and down-regulation of acute inflammation (e.g., activation of endothelial cells and lymphocytes), bone formation and remodeling, insulin secretion, and fever induction. The IL-1 family of cytokines shares an overall structural similarity (see, e.g.,

Priestle et al., Proc Natl Acad Sci USA 86, 9667-71 (1989)). IL-lalpha and beta are typically secreted by macrophages, and are derived by enzymatic cleavage of precursor proteins (pro-IL-1 alpha and pro-IL-1 beta), and exert their physiological effects by binding to IL-1 receptors. The amino acid and nucleotide sequences for the IL-1 cytokines are known and publically available for a number of species, including human. IL-1 alpha, as well as the other cytokines, are commercially available, e.g., from Pierce, Biosource, and Peprotech. The sequence for the human IL-1 alpha protein (precursor and mature form) is available as SwissProt accession number P01583.1, and the coding sequence is available as Genbank accession number AK314850.1.

IL-3, also know as multi-CSF, is a multilineage cell cytokine/growth factor secreted by lymphocytes, epithelial cells, and astrocytes, that stimulates the clonal proliferation and differentiation of various types of blood and tissue cells, particularly the differentiation and function of granulocytes and macrophages. It is considered a hematopoietic colony stimulating factor (see, e.g., Wagemaker et al., Biotherapy 2(4):337-45 (1990)). The amino acid and nucleotide sequences for IL-3 are known and publically available for a number of species, including human. Variants of IL-3 are described, e.g., in Lopez et al., Proc Natl Acad Sci USA 89:11842-6 (1992); Barry et al., J Biol Chem. 269:8488-92 (1994); and Olins et al., J Biol Chem. 270:23754-60 (1995)).

IL-6, also known as B-cell stimulatory factor 2 (BSF-2) and interferon-beta 2, is involved in regulating differentiation of B cells into immunoglobulin secreting cells, induction of myeloma/plasmacytoma growth, and nerve cell differentiation. IL-6 binding to IL-6 receptors induces formation of a multisubunit complex containing protein GP 130, which is common to the class I cytokine receptor superfamily. The amino acid and nucleotide sequences for IL-6 are known and publically available for a number of species, including human. Variants of IL-6 are described in Dagan et al., Protein Expr. Purif. 3:290-4 (1992); Zhang et al., Eur J Biochem. 207(3):903-13 (1992); and Skelly et al., J Biotechnol. 34:79-86 (1994). Recombinant forms are described in Stoyan et al., Eur J Biochem. 216:239-45 (1993); Orita et al., J Biochem (Tokyo) 15:345-50 (1994)). The protein and its variants are also commercially available.

IL-9 (also known as MCGF, MEA, megakaryoblast growth factor) is produced primarily by T cells and functions via binding the IL-9 receptor to stimulate proliferation and reduce apoptosis (see, e.g., Renauld et al. (1993) Adv. Immunol. 54:79). The amino acid and nucleotide sequences for IL-6 are known and publically available for a number of species, including human. The protein and its variants are also commercially available (e.g., Invitrogen, eBioscience, ProSpec).

IL-11 belongs to the IL-6 group of structurally and functionally related cytokines, which, as noted above, uses the transmembrane glycoprotein gp130 to exert its physiological activity. IL-11 is also known as adipogenesis inhibitor factor (AGIF) and oprelvekin. IL-11 acts synergistically with other cytokines and growth factors to stimulate proliferation and differentiation of stem cells into committed progenitor cells and to promote megakaryopoiesis and thrombopoiesis. The amino acid and nucleotide sequences for IL-11 are known and publically available for a number of species, including human. Recombinant forms and variants of IL-11 are described, e.g., in Miyadai et al., Biosci. Biotechnol. Biochem. 60:541-2 (1996); Tacken et al., Eur J Biochem. 265:645-55 (1999)).

IL-20 is a member of the IL-10 family of cytokines, and is produced primarily by activated keratinocytes and monocytes (Wahl et al. (2009) J. Immunol. 182:802). Binding to the IL-20 receptor results in activation of the STAT3 signaling pathway (Tohyama et al. (2009) Eur. J. Immunol. 39:2779). The amino acid and nucleotide sequences for IL-11 are known and publically available for a number of species, including human.

### V. Conditions Amenable to Treatment

The MKPs and/or megakaryocytes described herein can be administered to an individual to increase the number of platelets in the individual. Thus, the MKPs and/or megakaryocytes can be used to treat the conditions and disorders described below, and those that are characterized by a reduced number of platelets. In some embodiments, the MKPs and/or megakaryocytes described herein are administered to an individual with thrombocytopenia to increase the number of platelets in the individual, thereby treating the thrombocytopenia.

The term thrombocytopenia refers to any disorder in which there are not enough platelets, and when severe, can lead to severe morbidity and mortality. The condition is sometimes associated with abnormal bleeding. Thrombocytopenia can be divided according to three major causes: low production of platelets in the bone marrow, increased breakdown of platelets in the bloodstream, and increased breakdown of platelets in the spleen or liver. Disorders that involve low production in the bone marrow include aplastic anemia and cancer in the bone marrow. Disorders that involve the breakdown of platelets include: immune thrombocytopenic purpura (ITP), drug-induced immune thrombocytopenia, drug-induced nonimmune thrombocytopenia, thrombotic thrombocytopenic purpura, primary thrombocythemia, disseminated intravascular coagulation (DIC), hypersplenism, etc.

Thrombocytopenia can occur upon exposure to ionizing radiation, e.g., accidental or therapeutic radiation. Reduction in platelet numbers can occur with exposure to around 1-10 Gy. Chemotherapy-induced thrombocytopenia (CIT) can also occur after patients undergo myelosuppressive or myeloablative chemotherapy. This often leads to a reduction in the dose of the chemotherpeutic agent, and can negatively affect the treatment outcome. MKPs are particularly sensitive to chemotherapeutic agents, while CD34⁺ cells and mature megakaryocytes (MK) are less affected.

Occurrence of thrombocytopenia can also result from the impaired development of megakaryocytes, complications from infections, and in transplant situations, e.g., where a patient undergoing myoablative treatment receives hematopoietic stem cell (HSC) transplant. In this case, thrombocytopenia can result from delayed or low engraftment of HSCs and from graft versus host disease (GVHD). Managing thrombocytopenia is critical after any myoablative/myelotoxic treatment to minimize life-threatening complications.

Another complication that can arise from myoablative therapy is neutropenia, a condition characterized by abnormally low numbers of white blood cells, particularly neutrophils, which are short lived and represent the most abundant leukocyte in the peripheral blood. Both thrombocytopenia and neutropenia arise from impaired hematopoiesis and the inability of the hematopoietic system to adequately replenish the terminally differentiated myeloid cell associated with each disorder. Both can also develop from other causes of impaired hematopoiesis, such as unintended exposure to lethal doses of ionizing radiation, inherited immunodeficiencies, viral infections affecting the bone marrow, and metabolic disorders (e.g., vitamin deficiencies). One standard therapy for neutropenia is administration of G-CSF.

Combination therapies for treating both thrombocytopenia and neutropenia can be designed using the MKP cells described herein.

### VI. Therapeutic Applications

The MKPs and/or megakaryocytes described herein can be administered in a pharmaceutical composition that includes a pharmaceutically acceptable excipient (e.g., a saline solution, or other isotonic buffer solution). The MKPs and/or megakaryocytes are typically administered at a pharmacologically effective dose. The terms “therapeutically effective amount,” “pharmacologically effective amount,” or “therapeutically” or “pharmacologically effective dose” refer to the amount sufficient to produce the desired physiological effect , e.g., increasing platelet numbers in an individual, particularly for treating the disorder or disease (e.g., thrombocytopenia), including reducing or eliminating one or more symptoms or manifestations of the disorder or disease. For example, a therapeutically effective amount can refer to the number of cells required to increase platelet levels in an individual by at least 10%, e.g., at least 20, 30, 50, 75, 80, 100% or more, compared to a control (e.g., an individual suffering the same disorder but not receiving treatment, or the same individual prior to treatment). In some embodiments, the MKPs and/or megakaryocytes are administered in amount to increase patient survival.

The amount of the MKPs needed for achieving a therapeutic effect can be determined empirically in accordance with conventional procedures, and can vary widely as a function of the age, weight and state of health of the patient, the nature and the severity of the indication. In some embodiments, the numbers of MKPs infused ranges from 10⁴-10⁹ cells/kg, e.g., about 10⁵ to about 10⁷ cells/kg, or about 10⁵ cells/kg of body weight, or more as necessary.

Transplantation of MKP or megakaryocytes into an individual is accomplished by methods generally used in the art for administering hematopoietic cells, e.g., intravenous injection (bolus or infusion). As described above, the number of cells transfused will take into consideration factors such as sex, age, weight, the types of disease or disorder, stage of the disorder, the percentage of the desired cells in the cell population (e.g., purity of cell population), and the cell number needed to produce a therapeutic benefit.

Cells can be administered in one injection (infusion), or through successive injections over a defined time period sufficient to generate a therapeutic effect. A pharmaceutically acceptable carrier is used for infusion of the cells into the patient. The carrier will typically comprise, for example, buffered saline (e.g., phosphate buffered saline), unsupplemented cell culture medium, or medium as known in the art.

The following discussion of the invention is for the purposes of illustration and description, and is not intended to limit the invention to the form or forms disclosed herein. Although the description of the invention has included description of one or more embodiments and certain variations and modifications, other variations and modifications arc within the scope of the invention, e.g., as may be within the skill and knowledge of those in the art, after understanding the present disclosure. All publications, patents, patent applications, Genbank numbers, and websites cited herein are hereby incorporated by reference in their entireties for all purposes.

### VII. Examples

A. Materials and Methods

Isolation of human CD34⁺ cells. G-CSF mobilized peripheral blood was harvested from healthy donors via leukapheresis (see, e.g., Kawamoto et al. (2009) Stem Cells 27:2857). CD34+ cells were positively selected using magnetic beads (Dynal, Lake Success, N.Y.) linked to anti-CD34 monoclonal antibody on an Isolex Device (Baxter, Deerfield, Ill.). CD34⁺ cells were enriched to 96-98% purity. Aliquots of 15-20×10⁶ cells/ml were cryopreserved until use.

Expansion of CD34⁺ cells. CD34⁺ cells from donors were thawed, pooled as indicated, and seeded at 5×10⁵ cells/ml in serum-free X-vivo 15 medium (Lonza, Walkersville, Md.) supplemented with IX GlutaMAX (Invitrogen, Carlsbad, Calif.). Cytokines were added as follows. Unless otherwise indicated, 10 nM mimetic Thrombopoietin (AF 15705; Cwirla et al. (1997) Science 276:1696; Anaspec, San Jose, Calif.) was used, though recombinant human TPO (rhTPO) and various other mimetics were also tested with similar results. The active range for TPO mimetic was 0.1-100 nM. IL-1α (Peprotech, Rocky Hill, N.J.) was typically used at 10 ng/ml, with an active range 1-200 ng/ml tested. IL-9 was typically used at 60 ng/ml IL-9, with an active range of 1-200 ng/ml. HSA (purified human serum albumin protein) was typically used at 1%, with an active range of 0.2-4%. Cultures were incubated at 37° C. in a humidified atmosphere containing 5% carbon dioxide. Viable cells were counted daily and cell concentration was readjusted to about 5×10⁵ cells/ml by the addition of fresh medium with cytokines.

Analysis of MKPs. Cell surface marker expression was analyzed by flow cytometry using CD15b, CD33, CD34, CD41, CD42a, CD90, and CD184 antibodies. Prior to antibody staining, cells were washed with HBSS containing 2% BSA and blocked with rat and mouse IgG (SouthernBiotech, Birmingham, AL) for 10 minutes at 4° C. Cells were incubated for 30 minutes at 4° C. with following monoclonal antibodies: CD34-PE (Miltenyi Biotec, Auburn, Calif.), CD41a-APC, CD42a-FITC, CD33-PE-Cy7 (BD Pharmingen, San Jose, Calif.), CD15-biotin and streptavidin-PE-TxRed (eBioscience, San Diego, Calif.). Cells were washed with staining media and FACS analysis was performed on FACS Aria (BD Bioscience).

Colony formation assay. Quantitation of the colony forming potential of megakaryocytic progenitor cells was performed in Megacult-C® (Stem Cell Technologies, Vancouver, BC) following manufacturer instruction. Methyl cellulose assay was done in Methocult® (Stem Cell Technologies, Vancouver, BC) supplemented with the following cytokines: 10 ng/ml SCF, 10 ng/ml IL-3, 10 ng/ml IL-6, 10 ng/ml IL-I 1, 2 U/ml EPO, 10 nM TPO mimetic, 50 ng/ml GM-SCF, and 10 ng/ml Flt3L. Cells were plated at 500 cells/plate and colonies counted 12-14 days after seeding using an inverted microscope.

Cryopreservation of MKPs. Cells were harvested by centrifugation. Cryostor CS5 cryopreservation medium (Sigma-Aldrich, St. Louis, Mo.) was used to preserve the cells at a concentration of 2.5×10⁷ cells/ml. The cryopreservation media includes 5% DMSO but is devoid of animal products such as serum. Cells were stored in a Mister Frosty slow freezing device at −80° C. for 24 hr before being transferred to liquid nitrogen.

Transplantation of MKP in NSG mice. 7-9 week-old NSG (Nod SCID gamma) mice were obtained from Jackson Laboratory, Sacramento, Calif., housed in microisolators under pathogen-free conditions, and fed with autoclaved food and water. NSG mice are deficient for B, T, and NK cells, as well as several innate immunity components. Mice received a sublethal dose of 275 cGy total body irradiation using a Faxitron CP160 (Faxitron X-Ray, Lincolnshire, Ill.). Sulfamethoxazole/Trimethoprim-oral 800 mg/12 mg per IL (HiTech Pharmacal, Amityville, N.Y.) was added to the drinking water after mice were irradiated. Cryopreserved MKPs were thawed and viability assessed. The thawed cells were then injected directly into the mice anesthesized with isofluorane (Baxter, Deerfield, Ill.). The cells were injected retro-orbitally without further processing.

Human platelet detection in NSG mice. Peripheral blood (PB) was obtained via tail vein weekly. 10 μl of mouse blood was added to 90 μl of HBSS containing 1:10 Anticoagulant Sodium Citrate Solution 40 g/L (Baxter Fenwal, Lake Zurich, Ill.). Three-color flow cytometric analysis using directly conjugated monoclonal antibodies was performed. Antibodies used were mouse CD41 PE, human CD41 APC, human CD42a FITC (BD Pharmingen, San Jose, Calif.). PB was incubated with the antibodies for 20 minutes in the dark at room temperature. Counting beads (2.04 μm. Spherotech, Lake Forest, Ill.) and additional staining media were added to dilute the whole blood to ensure accurate platelet counts before analysis on a FACS Calibur (Becton Dickinson, San Jose, Calif.).

Human platelet activation with ADP. 20 μl of mouse blood was obtained from tail vein. 10 μl was added to 40 μl of HBSS+10% sodium citrate staining media (SM) (control). Another 10 μl of blood was added to 40 μl of 0.2 mM ADP (Bio/Data Corp, Horsham, Pa.) dissolved with SM. Whole blood in the two tubes was incubated for 10 minutes at room temperature. CD41 PE, CD42a FITC, and CD62P APC (BD Pharmingen, San Jose, Calif.) antibodies were added and incubated for 20 minutes in the dark at room temperature before analysis on FACS Calibur (Becton Dickinson, San Jose, Calif.).

Analysis of human engraftment in NSG mice. Mice were euthanized with carbon dioxide before tissue collection. Single-cell suspensions were prepared from the bone marrow and spleen. Flow cytometric detection of human cells in mice tissues was performed on FACS Aria (Becton Dickinson, San Jose, Calif.). The following directly-conjugated, anti-human antibodies were used: CD34-PE PE (Miltenyi Biotec, Auburn, Calif.), CD41 APC, CD42a FITC, CD33 PECy7, CD90 PE, CD71 PE, CD 19 APC, CD20 PE, CD3 PECy7 (all BD Pharmingen, San Jose, Calif.) CD15 biotin, CD2 biotin, streptavidin-PE-TxRed, streptavidin-PE (all eBioscience, San Diego, Calif.) and CD184 PECy7 (Biolegend, San Diego, Calif.). Cells were blocked with rat and mouse IgG (Southern Biotech, Birmingham, Ala.) for 10 minutes at 4° C. Cells were then incubated for 30 minutes at 4° C. Cells were washed and flow cytometric analysis was performed on a FACS Aria (Becton Dickinson, San Jose, Calif.).

B. Example 1: Generation of MKPs from CD34+ HSCs

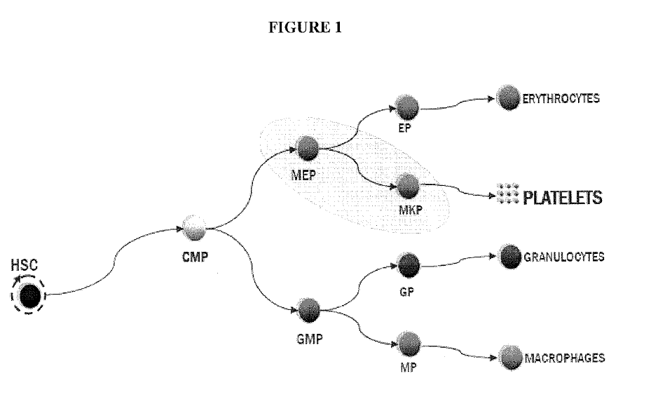

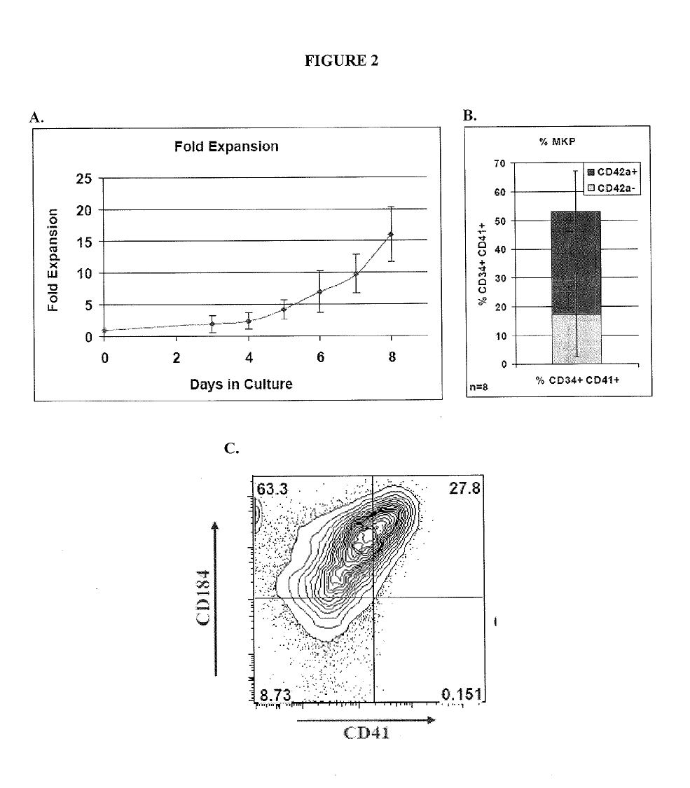

We identified culture conditions that generate about 40-70% CD34⁺ CD41⁺ megakaryocyte progenitor cells (MKPs) from CD34⁺ mobilized peripheral blood cells (FIGS. 1 and 2). 10 ng/ml of the TPO mimetic AF15705 and 10 ng/ml IL-1α were added to X-Vivo 15 media. These conditions are referred to as T1a or CLT-009 in the figures. The fold expansion from the starting population of CD34+ cells varied depending on donor, but ranged from 5-25 fold in 8 days of culture.

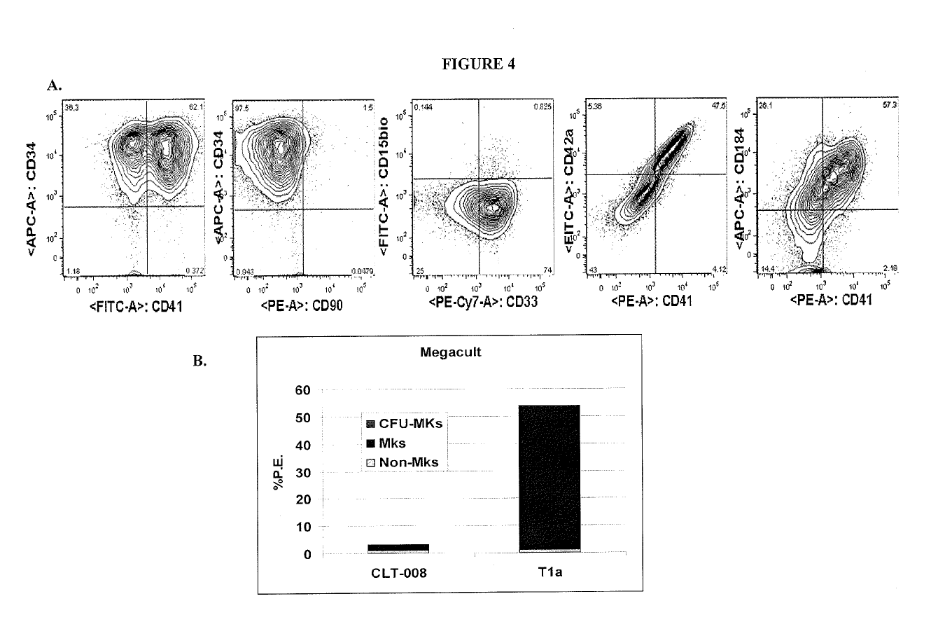

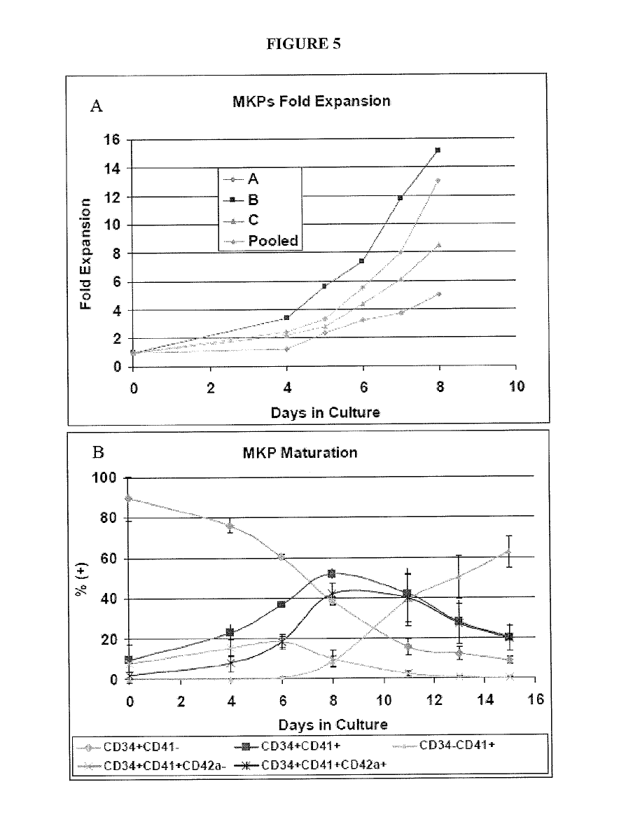

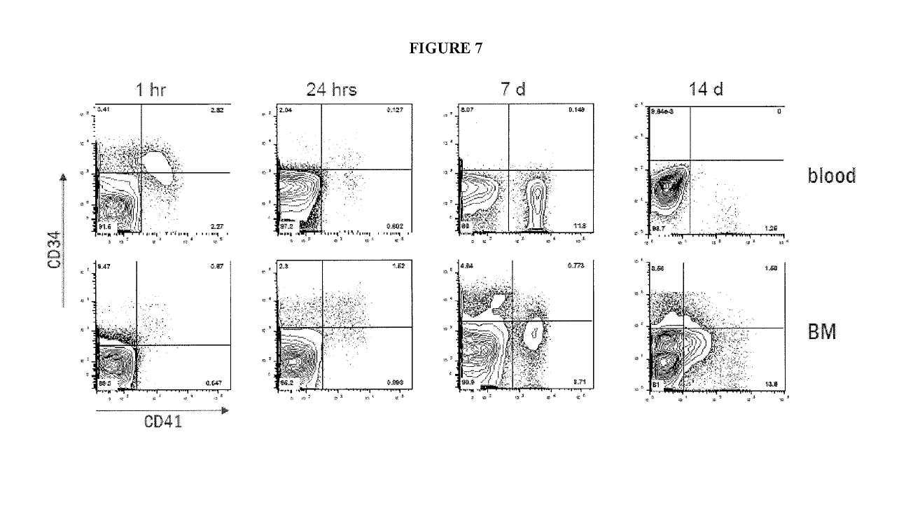

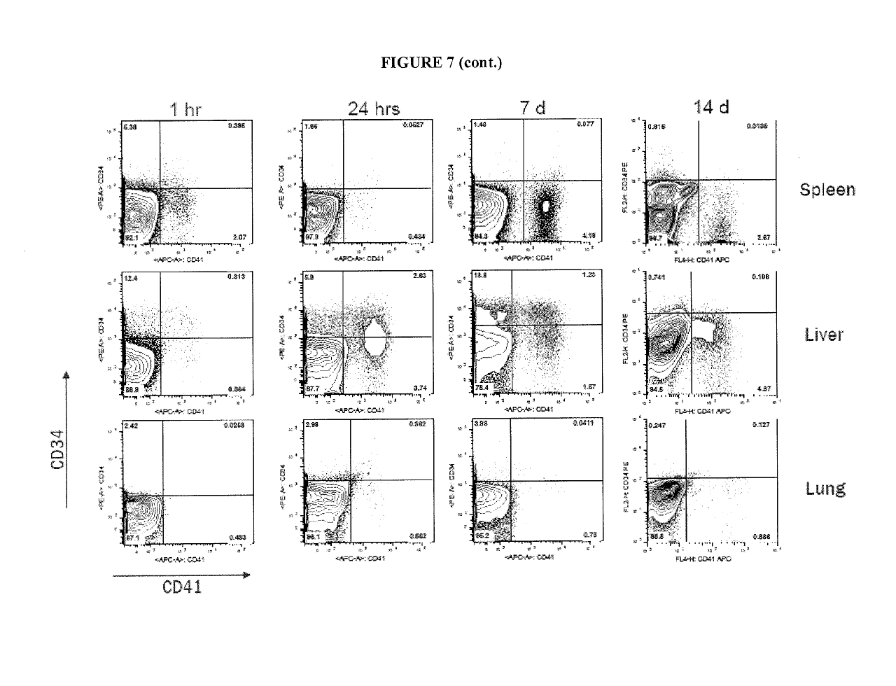

We found that addition of IL-3 to the growth media reduced expression of CD184 (CXCR4) on the MKPs. CD184 is involved in proper homing of the cells to the bone marrow, and thus can be involved engraftment of transplanted cells. FIG. 2C and FIG. 4A show that MKPs grown in TPO+IL-1alpha are largely CD positive. FIG. 7 shows that the expanded MKPs, upon administration, home to the bone marrow and further mature.

C. Example 2: MKPs are Predominantly Committed to the Megakaryocyte Lineage

To determine the stage of commitment of the MKPs generated in Example 1 (labeled CLT-009), we exposed the cells to conditions that promote either neutrophil or megakaryocyte differentiation, and compared development of the cells to that in a less differentiated myeloid progenitor population (CLT-008, see, e.g., WO2007095594). Day 8 MKPs were cryopreserved in Cryostor™ CS5 cryopreservation medium. CLT-008 cells were also cryopreserved prior to the neutrophil or megakaryocyte promoting culture.

Conditions for promoting neutrophil differentiation included 10 ng/ml SCF, 10 nM AF15705, 100 ng/ml Flt3-L, 10 ng/ml IL-3, 100ng/ml G-CSF, and 10% FBS. Cells were cultured for 6 days after reconstitution. Conditions for promoting megakaryocyte differentiation included 10 nM AF15705 30 10 ng/ml IL-1alpha. Again, cells were cultured for 6 days after reconstitution.

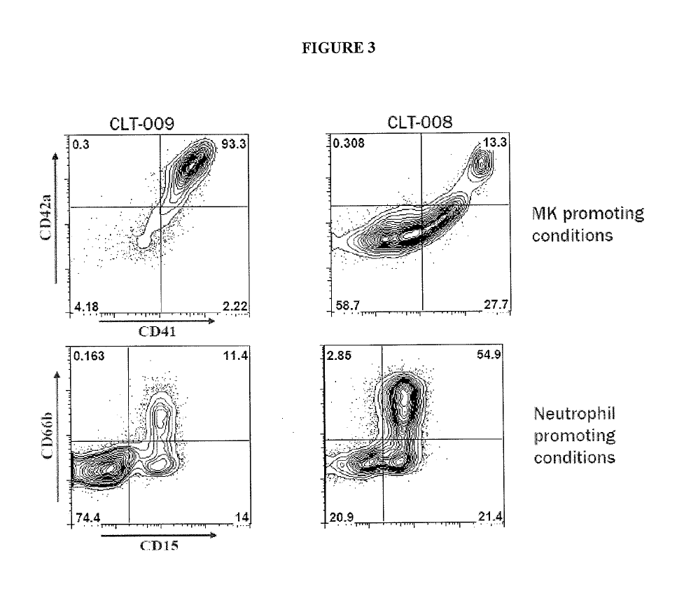

As shown in FIG. 3, the CLT-009 cells generated as in Example 1 produced large numbers of megakaryocytes (CD41+CD42a+), but relatively few neutrophils (CD15+CD66b+). The less differentiated myeloid progenitors, on the other hand, generated greater numbers of neutrophils relative to megakaryocytes (FIG. 3, right panels). The results indicate that the present MKPs are primarily committed to the megakaryocyte lineage.

Further evidence for the megakaryocyte character of the MKPs is shown in FIG. 4A. At Day 8 of culture, the cells are predominantly positive for CD33, CD34, CD41, and CD184. Later stage MKPs are also evident as a CD41+CD42a+ population. Expression of CD 15, a marker of the GMP lineage is largely negative, as is expression of the HSC marker CD90.

The results in FIG. 3 were further confirmed in a Megacult® assay, which promotes megakaryocyte growth. Megacult® medium with cytokines was obtained from Stem Cell Technologies (Vancouver, BC). The medium includes the following components in Isocoves MDM, 1.1 mg/mL Collagen, 1% Bovine Serum Albumin, 10 mg/uL rh Insulin, 200 ug/mL Human Transferrin (Iron saturated), 10⁻⁴M 2-Mercaptoethanol, 2 mM L-glutamine, 50 ng/mL rh Thrombopoietin, 10 ng/mL rh IL-6, and 10 ng/mL rh IL-3. While the myeloid progenitor cells generated relatively few megakaryocytes (FIG. 4B, left), the MKPs generated as in Example 1 (T1a) generated a significant number of megakaryocytes.

D. Example 3: Human MKPs Generated from CD34+ Peripheral Blood Cells in Defined Serum-Free and Feeder-Free Conditions

Expansion cultures of human peripheral blood CD34+ cells were performed in serum-free medium containing mimetic Thrombopoietin and IL-1α. FIG. 5a shows the growth kinetics of 3 human donors and the pool of the same three donors (pooled). The cultures yielded on average a 10±4.5 fold increase of total nucleated cells (TNC) after 8 days of culture. Donor A, B, C, and pooled expanded 5.15, 8.5, and 13-fold respectively (FIG. 5a). FIG. 5b shows the composition of the cell culture from day 0- day 16. The percentage of CD34⁺CD41⁻ progenitors decreased as they began to differentiate into early stage MKPs (CD34⁺CD41⁺CD42a^{neg}). Early stage MKPs began to decrease on day 6 to differentiate into late stage MKPs (CD34⁺D41⁺CD42a⁺). Late stage MKPs expanded ˜day 6 to day 8, and continued to mature. CD34 expression decreased and the cells increasingly matured into MKs (CD34-CD41+) after day 8.

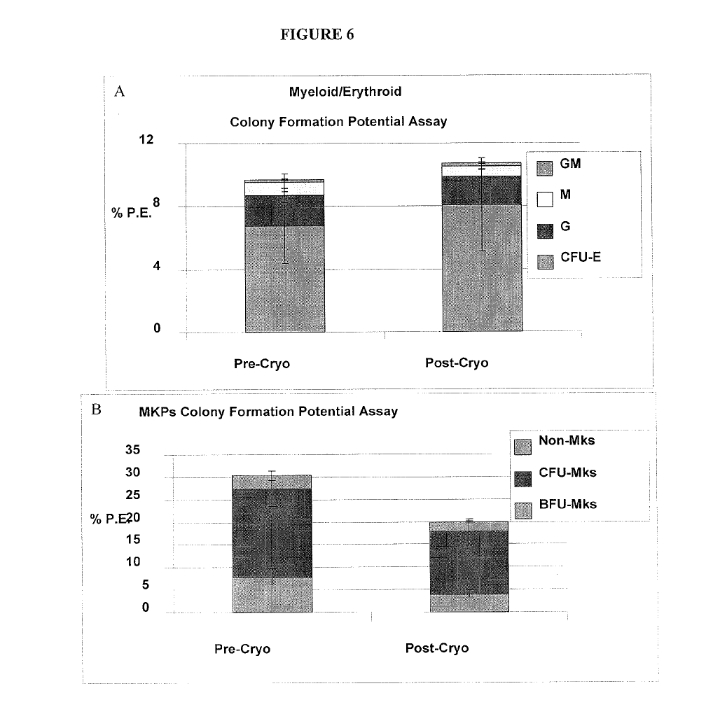

On average, the culture composition at day 8 was: 38.5±1.81% CD34⁺CD41⁻; 51.8±2.29% CD34⁺CD41⁺; and 8.41±2.84% CD34-CD41+. Some day 8 cells were cryopreserved for further analysis, while the rest were continued in culture until day 15. The MKP maturation profile in FIG. 6b show that the percentage of CD34+CD41+ was about 50% at day 8, indicating that the highest percentage of platelet producing MKPs can be obtained after about 8 days in the present culture conditions.

E. Example 4: MKPs Retain Colony Forming Potential after Cryopreservation

Platelets are known to rapidly lose viability over time in storage, at either 4° C. or freezing conditions (−80° C.) (see, e.g., Pence (2010) the website at healthnews.uc.edu/publications/findings/?/10843/10967 and Baldini et al. (1960) Blood 16:1669). To determine the effect, if any, of cryopreservation on the present MKP population, in vitro methyl cellulose colony formation potential assays were carried out as described above. MKPs taken from day 8 culture generated about 10% myeloid colony forming units, with about 6% erythroid colony forming progenitors (FIG. 6A, left). After cryopreservation, MKP did not lose colony formation potential (FIG. 6A, right).

To determine megakaryocyte colony forming potential from these cells, progenitor quantification assays were performed in Megacult®. Unfrozen day 8 MKPs generated approximately 7.9% BFU-MKs and 19.6% CFU-MK with few non-MK colonies. Post-cryopreservation Megacult® assays showed a slight decrease in megakarycyte colony formation potential (FIG. 6B).

F. Example 5: MKPs Continue to Mature and Generate Platelets in Vivo after Cryopreservation

Day 8 post-cryopreservation MKPs (10⁷) were injected into NSG mice as described above. Engraftment of the cells in bone marrow and the other tissues indicated in FIG. 7 was determined 1 hour, 24 hours, 7 days, and 14 days post-administration to determine localization of the MKPs. Results show that MKPs migrated to the bone marrow as soon as one hour after transplantation, and are largely absent from peripheral blood within 24 hrs.

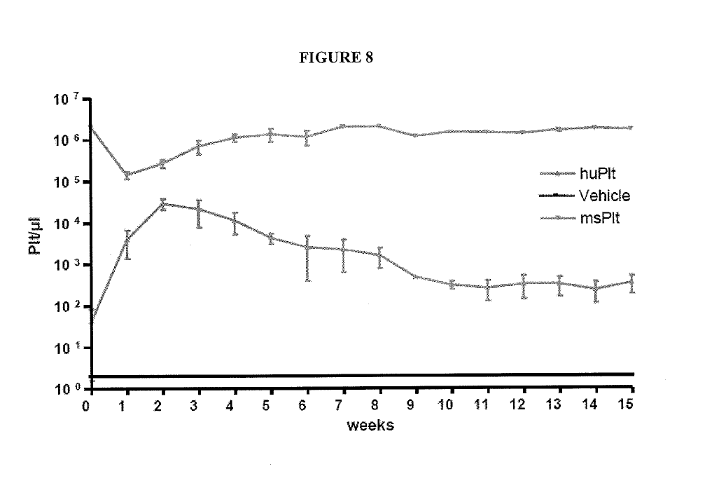

FIG. 7 shows the localization of CD34⁺CD41⁺ cells (MKPs). The figure also shows that the cells continue to mature. CD34 expression is reduced to produce a CD34⁻CD41⁺ population by Day 7. Human platelets were detectable 3 days after injection, and platelet numbers peaked on about Day 14 at about 3×10⁴ Plt/ul (FIG. 8). Human platelets were observed at least 8 weeks post-injection.

G. Example 6: Day 8 Post-Cryopreservation MKPs Rapidly Generate High Platelet Numbers

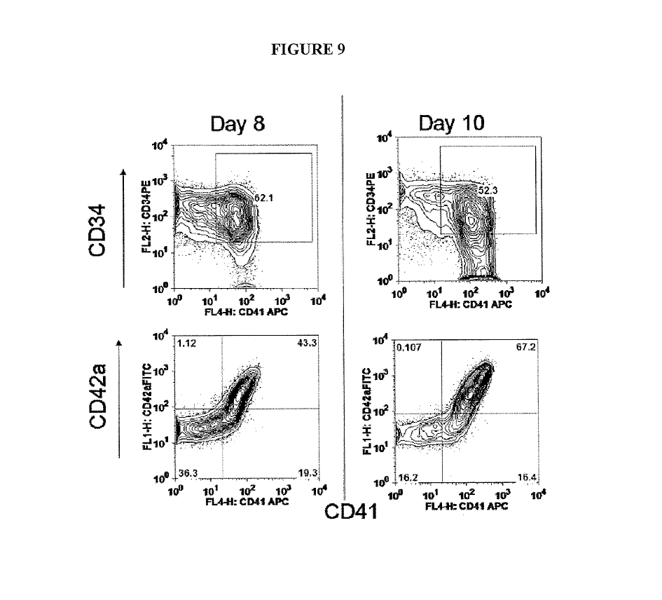

We sought to isolate the MKP population that yields high platelet numbers in a short time period in vivo. 10⁷ MKPs, cultured as described above for 8 or 10 days, cryopreserved and thawed, were injected into NSG mice (n=4). CS5 cryopreservation media was injected in the control group. FIG. 9 shows results for post-cryopreservation Day 8 and Day 10 MKPs. Day 8 MKPs had a higher percentage of CD34+CD41+ cells than Day 10 MKPs. The Day 10 MKPs had less CD34⁺CD41⁺ and more CD41⁺CD42a^{±}population (FIG. 9, right).

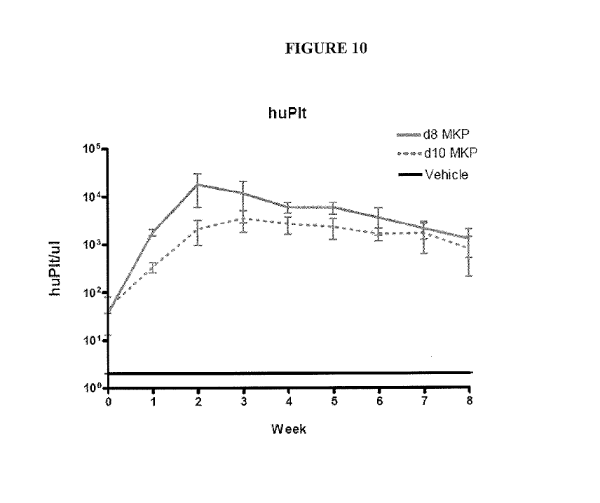

Platelet numbers are shown in FIG. 10. Human platelets can be detected as early as 3 days post-administration for both Day 8 and Day 10 MKPs. Day 8 MKPs, however, generated higher platelet levels (2×10⁴ plt/ul), which peaked sooner (week 2). Day 10 MKP generated 1×10³ plt/ul on week 2, and peaked at about week 3 at 3×10³ plt/ul.

H. Example 7: Human Platelets Generated by the Present Post-Cryopreserved MKPs are Functional

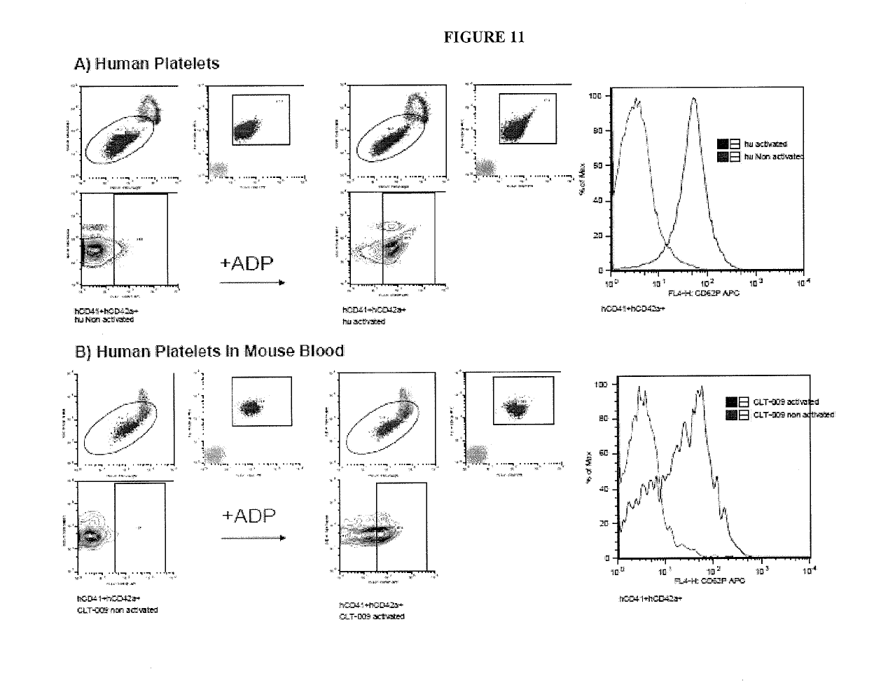

In vitro platelet activation assays were performed to determine the functionality of platelets generated from human MKPs in vivo in the xenograft NSG mice. Human blood, which includes normal human platelets, was used as a control for whole blood from the xenograft NSG mice. Platelets were exposed to ADP as described above. The translocation of P-Selectin (CD62P) to the platelet surface was used to detect activation of platelets in response to the ADP stimulus. FIG. 11 shows that platelets from both freshly isolated human whole blood and freshly isolated xenograft mouse whole blood expressed CD62P, indicating normal platelet activation.

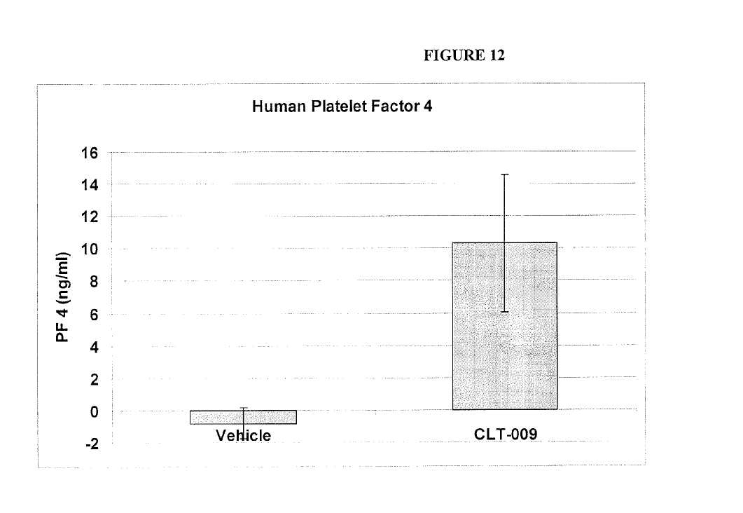

Blood was obtained on Day 14 from mice receiving 10⁷ CLT-009 cells. Platelets were exposed to ADP for activation as described above. Supernatant was tested by the ELISA for release of platelet factor 4 (PF4). The result shown in FIG. 12 reveals that the human platelets generated from CLT-009 product release PF4 upon activation like naturally occurring platelets.

I. Example 8: Generation of MKPs is Increased in the Presence of IL-9

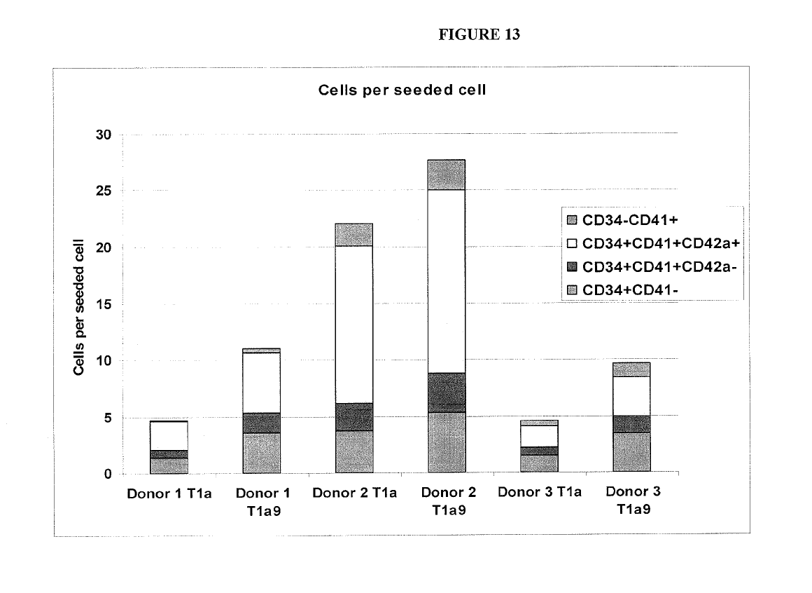

MKPs were cultured for 8 days as described above with TPO mimetic and IL-1 in the presence or absence of various concentrations of IL-9. FIG. 13 shows results for IL-9 at 60 ng/ml. IL-9 increased the number of MKPs from all 3 donors, including the potent CD34+CD41+ populations.

J. Example 9: Generation of MKPs is Increased in the Presence of HSA, and with Agitation

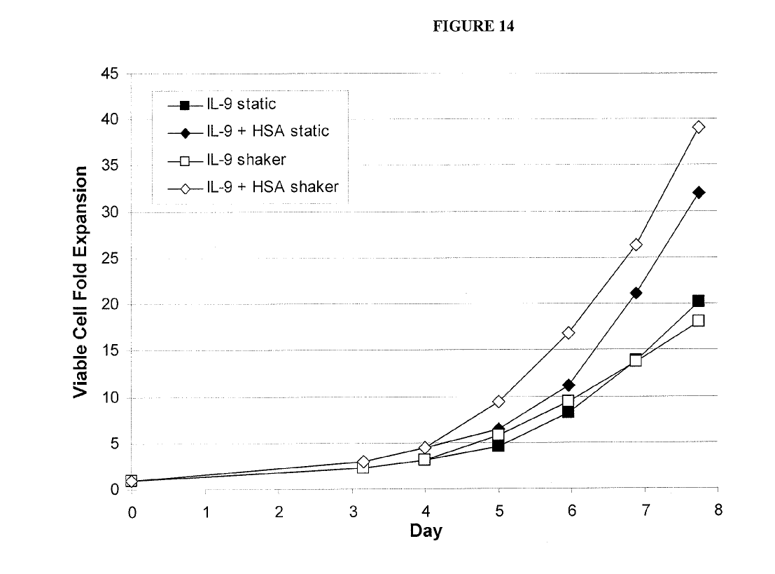

MKPs were cultured for 8 days as described above with TPO mimetic, IL-1, and IL-9 in the presence or absence of various concentrations of HSA. FIG. 14 shows the results for 1% HSA, which further increased MKP cell expansion. In addition, cell numbers were compared in static culture vs agitated culture (in flasks). The data show that agitation in the presence of HSA increased MKP cell expansion considerably. Agitation allows for culture of larger volumes, and scaling up of production methods.

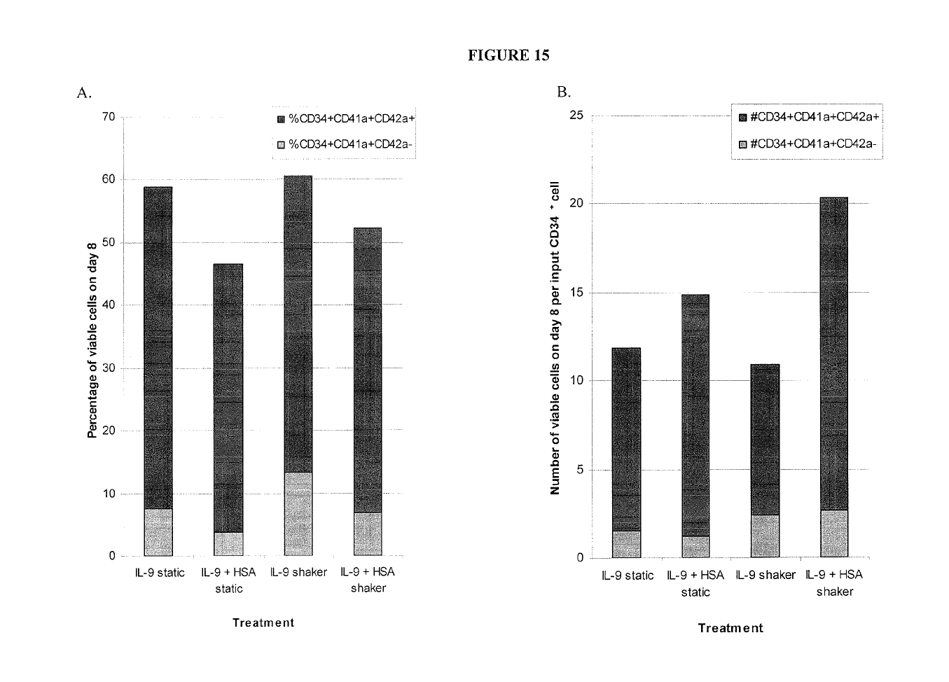

FIG. 15 shows the phenotypic analysis of the cell populations shown in FIG. 14. (A) shows that HSA leads to a slight reduction in the percentage of CD34+CD41+ cells. (B) demonstrates that, when the total number of cells is accounted for, HSA increases the total yield of CD34+CD41+ cells. Agitation of the HSA-containing culture increased cell numbers even more (compared to static culture bags).

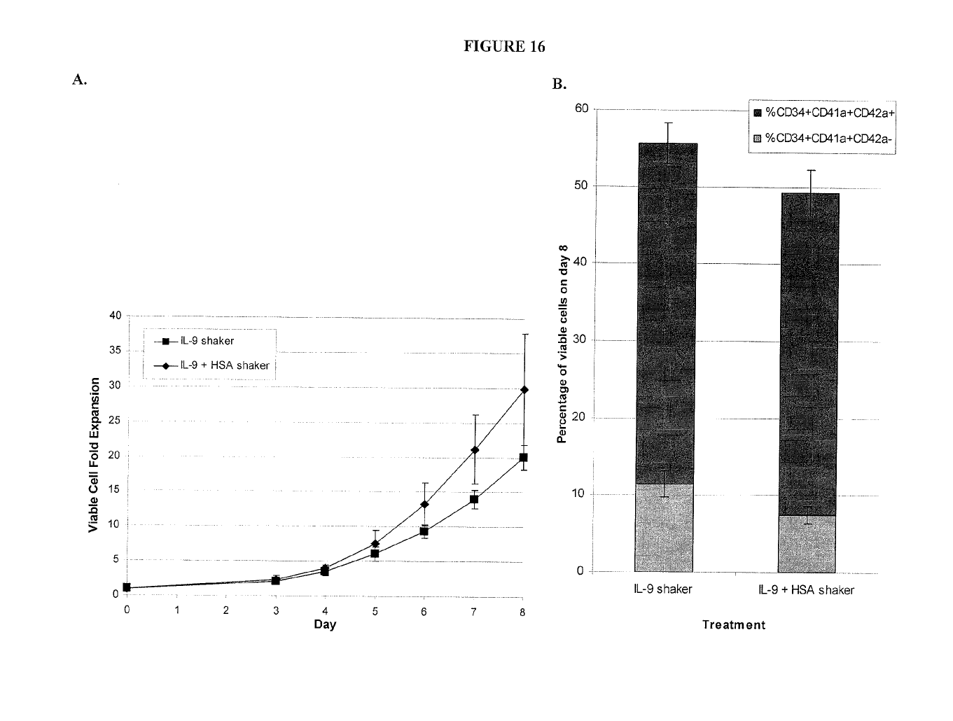

FIG. 16 shows the average fold expansion of three individual MKP cultures over eight days when cultured in the presence of 10 nM mTPO, 10 ng/m1 IL-1, 60 ng/ml IL-9 with or without additional 1% HSA. Cell numbers increased significantly in the presence of HSA (A) and the CD34+CD41+ population was maintained (B).

K. Summary

MKPs can be generated ex vivo under defined, serum-free and feeder-free culture conditions. The present MKPs, cultured as described and cryopreserved, maintain colony formation ability in vitro, and engraft and generate platelets in vivo. Moreover, the MKPs can be expanded in shaker flasks, allowing for larger culture volumes and commercial-scale production. Cryopreserved MKPs transplanted into mice migrated to the bone marrow and generated platelets within three days after transplant. MKPs continued to mature in vivo and consistently generated platelets over at least 8 weeks. The transplanted human MKPs generated platelets in mice that are functional, as assessed by ADP activation.

The presently described culture system is defined, scalable, and suitable for clinical use. The final population does not contain detectable levels of lymphoid cells and thus avoids the risk of graft versus host disease (GVHD). Patients suffering from acute radiation syndrome (ARS) or chemotherapy-induced thrombocytopenia (CIT) can thus greatly benefit from transplant of the presently described MKP population. These MKPs generate peak levels of platelets in vivo about 2 weeks post-administration, which can thus shorten the duration of thrombocytopenia in ARS or CIT. The issues associated with platelet transfusion such as short storage time, shortage of supplies, and risks of infectious disease can be overcome by providing a storable off-the-shelf MKP product.

1. An isolated population of human cells comprising at least 40% CD34+CD41+ megakaryocyte progenitor cells (MKPs), wherein said population of cells has platelet generating activity, and wherein administration of 5×10⁶ cells from the population of cells into an immunocompromised mouse results in at least 10⁶ human platelets/ml blood 14 days post-administration.

2. The isolated population of cells of claim 1, comprising at least 50% CD34+CD41+ MKPs.

3. The isolated population of cells of claim 1, wherein at least 50% of the CD34+CD41+ MKPs are CD184+.

4. The isolated population of cells of claim 1, wherein less than 60% of the CD34+CD41+ MKPs are CD42a+.

5. (canceled)

6. The isolated population of cells of claim 1, wherein the cells are derived from a plurality of individuals.

7. The isolated population of cells of claim 1, wherein the cells are cryopreserved.

8. The isolated population of cells of claim 1, wherein administration of 5×10⁶ of the cells into an immunocompromised mouse results in at least 10⁷ human platelets/ml blood 14 days post-administration.

9-16. (canceled)

17. A pharmaceutical composition comprising the isolated population of cells of claim 1 and a pharmaceutically acceptable excipient.

18. The pharmaceutical composition of claim 17, wherein the composition is cryopreserved.

19. A method comprising:
culturing human CD34+ hematopoietic stem cells (HSCs) in media comprising thrombopoietin (TPO) or a TPO analog and IL-1 a under conditions that permit the CD34+ HSCs to generate a population of cells comprising CD34+CD41+ megakaryocyte progenitor cells (MKPs),
wherein administration of 5×10⁶ cells from the population cells into an immunocompromised mouse results in at least 10⁶ human platelets/ml blood 14 days post-administration, thereby forming a population of cells comprising MKPs.

20. A method comprising:
culturing human CD34+ hematopoietic stem cells (HSCs) in media comprising thrombopoietin (TPO) or a TPO analog, IL-1α or IL-1β, and IL-9 under conditions that permit the CD34+ HSCs to generate a population of cells comprising CD34+CD41+ megakaryocyte progenitor cells (MKPs),
wherein administration of 5×10⁶ cells from the population cells into an immunocompromised mouse results in at least 10⁶ human platelets/ml blood 14 days post-administration, thereby forming a population of cells comprising MKPs.

21. The method of claim 19, wherein the media further comprises HSA.

22. The method of claim 19, wherein the culturing is carried out in agitation conditions.

23. The method of claim 19, wherein the media does not include serum or feeder cells.

24. The method of claim 19, wherein the HSCs are obtained from a plurality of individuals.

25. The method of claim 19, wherein the HSCs have been cryopreserved.

26-41. (canceled)

42. A method comprising
administering to an individual the pharmaceutical composition of claim 17, wherein the administering generates platelets in the individual.

43-44. (canceled)

45. The method of claim 20, wherein the media further comprises HSA.

46. The method of claim 20, wherein the culturing is carried out in agitation conditions.

47. The method of claim 20, wherein the individual and the MKPs have a mismatch at one or more MHC loci.

48. The method of claim 20, wherein the HSCs are obtained from a plurality of individuals.

49-53. (canceled)
